(19) United States
(12) Patent Application Publication
(10) Pub. No.: US 2026/0201418 A1
(43) Pub. Date: Jul. 16, 2026

(54) ADENOVIRAL HELPER PLASMID

(71) Applicant: Forge Biologics, Inc., Grove City, OH (US)

(72) Inventors: Angela M. Adsero, Groveport, OH (US); Brendan Chestnut, Groveport, OH (US); Linas Padegimas, Mayfield Heights, OH (US); David Dismuke, Cary, NC (US)

(21) Appl. No.: 19/130,904

(22) PCT Filed: Nov. 16, 2023
(86) PCT No.: PCT/US2023/080063

§ 371 Date: May 16, 2025

Related U.S. Application Data
(60) Provisional application No. 63/426,028, filed on Nov. 16, 2022.
(60) Provisional application No. 63/596,201, filed on Nov. 3, 2023.

(51) Int. Cl. C12N 15/864; C12N 7/02; C12N 15/861

(52) U.S. Cl. CPC C12N 15/8645; C12N 7/025; C12N 15/8613; C12N 2710/10044; C12N 2750/14143; C12N 2750/14144; C12N 2750/14152; C12N 2800/10; C12N 2830/34; C12N 2830/50

(57) ABSTRACT

The present disclosure provides improved adenoviral helper plasmids for the production of recombinant adeno-associated viruses.

### CROSS REFERENCE TO RELATED APPLICATIONS

This application claims priority to and the benefit of U.S. Provisional Patent Application Nos. 63/426,028, filed on Nov. 16, 2022 and 63/596,201, filed on Nov. 3, 2023, the entire contents of which are hereby incorporated by reference in their entirety.

### BACKGROUND

Adeno-associated virus (AAV) technology has quickly become a dominant form of gene therapy for genetic diseases. AAVs can be produced in large scale in a variety of host cell systems, including mammalian cells, such as HEK293 cells. Traditionally, AAV production in mammalian cells involves the introduction of multiple plasmids to the host cells, the plasmids encoding, for example, a human gene or genes of interest, and various viral genes critical for viral replication and packaging. Due to the number of genes required for proper replication, these are traditionally delivered on two or three separate plasmids.

One such plasmid, termed an “adenoviral helper” plasmid, contains genes critical for AAV production from a host cell. Adenoviral helper plasmids containing E2a, VA RNA, and E4 genes have been shown to be critical to promoting AAV production in mammalian host cell systems.

Despite much advancement over the last two decades, concerns regarding the cost and safety of AAV production continue to limit the therapeutic potential of AAV technology. These concerns are due in part to the large size of many helper plasmids, which is due to the provision of a large number of genes on a single helper plasmid to support AAV production. The safety concerns are due in part to the production, albeit at low levels, of potentially cytotoxic and/or inflammatory viral proteins that are not necessary for AAV replication.

### SUMMARY

In some embodiments, the present disclosure, provides, among other things, an adenoviral helper plasmid. In some embodiments, the present disclosure provides an adenoviral helper plasmid with reduced size relative to those known in the art. In some embodiments, the present disclosure provides an adenoviral helper plasmid comprising nucleotide sequences encoding E2a, VA RNA. E4; and an L4 region. In some embodiments, an adenoviral helper plasmid as described herein comprises nucleotide sequences encoding proteins from other viruses. In some embodiments, an adenoviral helper plasmid as described herein comprises nucleotide sequences encoding proteins from other viruses, including HSV-1 UL30, HSV-1 UL42, and/or HSV-1 UL29.

In some embodiments, the present disclosure provides an adenoviral helper plasmid that does not comprise one or more nucleotide sequences encoding one or more of fiber protein; L1-52/55K (Packaging Protein 3), peripentonal Hexon-Associated protein, and an L4 region. In some embodiments, the present disclosure provides an adenoviral helper plasmid comprising a fragment, portion, or partial form of E2a protein, VA RNA, E4, and an L4 region. In some embodiments, the present disclosure provides an adenoviral helper plasmid that does not comprise one or more nucleotide sequences encoding one or more of Hexon Associated Precursor (L4 pVIII) protein, DNA Terminal Protein, and 23 kDa endoprotease. In some embodiments, the present disclosure provides an adenoviral helper plasmid that does not comprise one or more nucleotide sequences encoding one or more of E4orf1, E4orf2, E4orf3, and E4orf7. In some embodiments, an adenoviral helper plasmid provides herein comprises a kanamycin resistance gene.

In some embodiments, the present disclosure provides an adenoviral helper plasmid in which expression of E2a protein is under the control of one or more of an E2a promoter, chicken β-actin promoter, and SV40 promoter. In some embodiments, the present disclosure provides an adenoviral helper plasmid in which expression of E4 open reading frames (ORFs) is under the control of one or more of a native E4 promoter and SV40 promoter.

In some embodiments, the present disclosure provides an adenoviral helper plasmid comprising a nucleotide sequence that is at least 80% identical to SEQ ID NO: 1-3, 5, 7, 9, 11-12, 14-20, 22, 24, 26-29, 31, 33, 35-37, 39-53, 56, 59, 62, 65, 68 or 80-105. In some embodiments, the present disclosure provides an adenoviral helper plasmid comprising a nucleotide sequence that encodes for an amino acid sequence that is at least 80% identical to SEQ ID NO: 4, 6, 8, 10, 13, 21, 23, 25, 30, 32, 34, 38, 55, 58, 61, 64, 67, or 69. In some embodiments, the present disclosure provides an adenoviral helper plasmid comprising a nucleotide sequence that is at least 80% identical to any one of SEQ ID NO: 41-49, 70, or 80-105.

In some embodiments, the present disclosure provides for provision of the L4 region in trans relative to an adenoviral helper plasmid. In some embodiments the present disclosure provides an L4 trans plasmid comprising a nucleotide sequence encoding an engineered L4 region. In some embodiments, an L4 trans plasmid encodes for an L4 region (e.g., L4 33K and L4 22K. In some embodiments, an L4 trans plasmid encodes for L4 33K. In some embodiments, an L4 trans plasmid encodes for L4 22K. In some embodiments the present disclosure provides a composition comprising an adenoviral helper plasmid described herein and an L4 trans plasmid described herein.

In some embodiments, the present disclosure provides methods of producing recombinant adenoviral associated viral vectors (rAAV). In some embodiments, a method of producing a rAAV comprises transfecting a producer cell with an AAV vector plasmid, and an adenoviral helper plasmid as described herein. In some embodiments a method of producing a rAAV comprises transfecting a producer cell with an AAV vector plasmid, an AAV Rep-Cap expressing plasmid, and an adenoviral helper plasmid described herein. In some embodiments a method of producing a rAAV further comprises transfecting a producer cell with an L4 trans plasmid. In some embodiments, a producer cell stably expresses Rep-Cap. In some embodiments, a producer cell comprises a nucleotide sequence encoding an L4 region or portion thereof (e.g., L4 33K and/or L4 22K).

### BRIEF DESCRIPTION OF THE DRAWING

FIG. 1. shows a plasmid map illustrating adenoviral helper plasmid pEMBR-1.2.

FIG. 2. shows vector yields obtained using pEMBR-1.2 and commercially available pX80 as the adenoviral helper plasmid.

FIG. 3. shows vector transgene purity and vector capsid purity obtained using pEMBR-1.2 or commercially available pX80 as the adenoviral helper plasmid.

FIG. 4. shows a comparison between the GFP expression levels obtained following the transformation of HEK293 cells with recombinant AAV RH.10, ssCMV-GFP transgene, and either pX80 or pEMBR helper plasmid.

FIG. 5. shows a plasmid map illustrating adenoviral helper plasmid pEMBR-1.3 and pEMBR-1.3B.

FIG. 6. shows a plasmid map illustrating adenoviral helper plasmid pEMBR-1.4 and pEMBR-1.4B.

FIG. 7. shows a plasmid map illustrating adenoviral helper plasmid pEMBR-1.5.

FIG. 8. shows a plasmid map illustrating adenoviral helper plasmid pEMBR-1.5A.

FIG. 9. shows an exemplary sequence map of native Adenovirus Type 5 genome including, among other things, VA RNA, E2a, and E4 regions (Panel A). Panels B, C, and D show the approximate sizes from pEMBR-1.2B2 of our VA RNA region (Panel B), E2a region (Panel C), and E4 region (Panel D) in comparison to the region from Matsushita 1998 annotated on the native Adenovirus sequence.

FIG. 10. shows an exemplary plasmid map containing VA RNA, E2a, and E4 regions, for example, as defined by restriction enzyme sites, e.g., as in plasmids described herein.

FIG. 11. shows a sequence map illustrating E2a region of pEMBR-1.2B2.

FIG. 12. shows sequences maps for various promoters upstream of E2a regions within various pEMBR plasmids as described herein.

FIG. 13. shows sequence maps for various truncations to the NotI end of the pEMBR1.2B2 E2a region, with one truncation construct having the addition of a promoter, as described herein.

FIG. 14. shows a table summarizing plasmid size (bp), VA RNA region modifications, E2a region modifications, and E4 region modifications in various pEMBR plasmids as described herein.

FIG. 15. shows average AAV9:eGFP titer (VG/mL) as measured by qPCR (Panel A) and ddPCR (Panel B) and protein expression of Rep proteins and E2a as measured by Western Blot (Panel C) using various pEMBR plasmids as described herein.

FIG. 16. shows average AAV9:eGFP titer (VG/mL) as measured by qPCR using various pEMBR plasmids as described herein.

FIG. 17. shows a table summarizing the inclusion of JEP, GEP, and native L4 (33K & 22K) and expression of E2a protein and production of AAV in various pEMBR plasmids as described herein.

FIG. 18. shows a table summarizing plasmid size (bp), VA RNA region modifications, E2a region modifications, and E4 region modifications in various pEMBR plasmids as described herein.

FIG. 19. shows a table summarizing plasmid size (bp), VA RNA region modifications, E2a region modifications. E4 region modifications, and AAV production relative to pEMBR-1.2.B2 in various pEMBR plasmids as described herein.

FIG. 20. shows sequence maps for native E4 region (Panel A) and SV40 promoter driven E4 ORF4 and E4 ORF6 with SV40 poly(A) signal (Panel B) (e.g., as present in pEMBR-1.7B2).

FIG. 21. shows a sequence map illustrating E2a region of pEMBR-1.2B2. The native L4 region (circled) illustrates sequences for 33 k and 22 k protein, contains 50 bp upstream of native promoter, and the presumably native poly(a) signal sequence. The L4 expression construct shown below reserves all the same features as the native L4 region except the native polyA signal has been replaced with a synthetic 49 bp polyA signal sequence.

FIG. 22. shows average AAV9:eGFP titer (VG/mL) using various pEMBR plasmids as described herein.

FIG. 23. shows average AAV titer compared to pEMBR-1.2B2 titer and plasmid size (bp) using various pEMBR plasmids as described herein.

FIG. 24. shows a plasmid map illustrating adenoviral helper plasmid pEMBR-1.37B2+L4-minKanR-v2.

FIG. 25. shows a sequence map for an exemplary E2a region which has been engineered to reduce plasmid size while maintaining AAV production.

FIG. 26. shows the location of the sequences reducing the E2a region in comparison to native Adenovirus Type 5 genome.

FIG. 27 shows a wild-type AAV E2a region with the additional depiction of the L4 region ORFs for 22K and 33K protein. The partial JEP sequence is depicted below JEP by the smallest open arrow, to draw attention to how this natural E2a promoter sequence can be added plasmids described herein when the L4 33K protein is added.

FIG. 28A and FIG. 28B show rescue of AAV titer by L4 region addition. FIG. 28A shows average rAAV titer (VG/mL) from triplicate 75 mL quadruple plasmid transfected cultures determined by qPCR for the pEMBR-1.3 plasmid containing the partial JEP (PJ) E2a construct plus the pUC57 vector containing the specified L4 construct in comparison to wild type plus empty vector (pEMBR-1.2B2+pUC57) production. The Partial JEP construct is shown as a reference. FIG. 28B shows Rep and E2a western blots of RIPA lysates from transfected cultures in 28A.). Beta-actin is shown as a loading control. Adenovirus type 5 infected HEK293/HEK293T cells are shown as a positive control and uninfected/nontransfected HEK293 cells are shown as a negative control.

FIGS. 29A, 29B, and 29C show increase of AAV titer by provision of additional copies of L4 and L33K. FIG. 29A shows the E2a regions of the helper plasmids used in quadruple plasmid transient transfection. FIG. 29B shows the average rAAV titer (VG/mL) from triplicate 75 mL quadruple plasmid transfected cultures determined by qPCR for the E2a constructs indicated in the figure plus the pUC57 vector containing the specified L4 construct to compare L4 gene dosage of 1X L4 genes, 2X L4 genes, 1X 33K with 2X 22K, and 2X 33K with 1X 22K against the control condition (wild type plus empty vector; 1.2B2+pUC57). FIG. 29C shows the Rep and E2a western blots of RIPA lysates from transfected cultures in FIG. 29A. Beta-actin is shown as a loading control. Adenovirus type 5 infected HEK293/HEK293T cells are shown as a positive control and uninfected/nontransfected HEK293 cells are shown as a negative control.

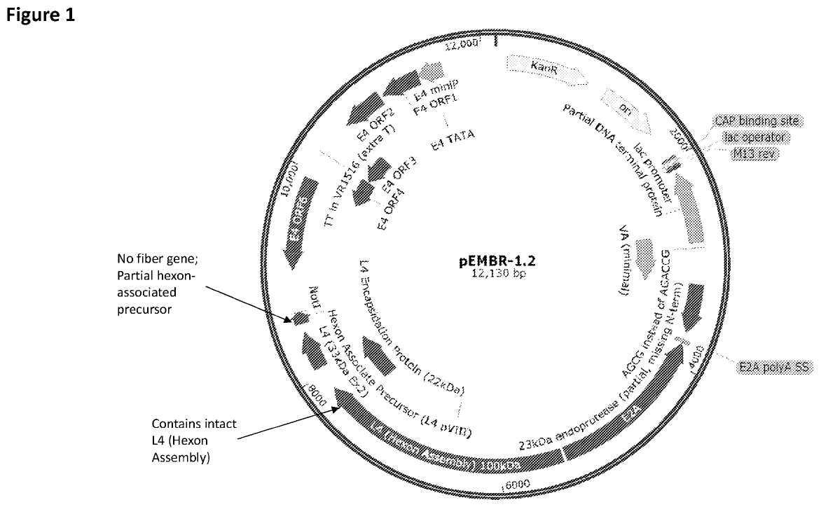

### DEFINITIONS

Agent: In general, the term “agent”, as used herein, is used to refer to an entity (e.g., for example, a lipid, metal, nucleic acid, polypeptide, polysaccharide, small molecule, etc, or complex, combination, mixture or system [e.g., cell, tissue, organism] thereof), or phenomenon (e.g., heat, electric current or field, magnetic force or field, etc). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Approximately/about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%. 2%, 1%, or less in either direction (greater than or less than) of the stated reference value unless otherwise stated or otherwise evident from the context (except where such number would exceed 100% of a possible value).

Comparable: As used herein, the term “comparable” refers to two or more agents, entities, situations, sets of conditions. etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances. individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA. GGSEARCH/GLSEARCH, Genoogle, HMMER. HHpred/HHsearch, IDF, Infernal, KLAST, USEARCH, parasail, PSI-BLAST, PSI-Search. ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Downstream: As used herein, the term “downstream” refers to the location or position of a nucleic acid sequence relative to a reference nucleic acid sequence, particularly a position that, during RNA transcription, is closer to the 3′ end of the transcribed RNA molecule encoded by the reference sequence. For example, for two sequences, A and B, such that sequence A is downstream of sequence B, transcription of sequence B proceeds toward sequence A.

Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments. “nucleic acid” refers to an individual nucleic acid residue (e.g., a nucleotide and/or nucleoside): in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is. comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine. C5-fluorouridine, C5-iodouridine. C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose. 2′-deoxyribose, arabinose. and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source. enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: As used herein, the term “operably linked” refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a distance from the functional element of interest.

Producer cell: As used herein, the term “producer cell” refers to any cell used to produce recombinant AAV (rAAV). In some embodiments, a producer cell is a mammalian cell. In some embodiments, a producer cell is a transformed mammalian cell. In some embodiments, a producer cell is a Vero, HeLa, HEK293, HEK293T cell or derivative thereof.

Transformation: As used herein, the term “transformation” refers to any process by which exogenous DNA is introduced into a host cell. Transformation may occur under natural or artificial conditions using various methods well known in the art. Transformation may rely on any known method for the insertion of foreign nucleic acid sequences into a prokaryotic or eukaryotic host cell. In some embodiments, a particular transformation methodology is selected based on the host cell being transformed and may include, but is not limited to, viral infection, electroporation, mating, lipofection. In some embodiments, a “transformed” cell is stably transformed in that the inserted DNA is capable of replication either as an autonomously replicating plasmid or as part of the host chromosome. In some embodiments, a transformed cell transiently expresses introduced nucleic acid for limited periods of time.

Upstream: As used herein, the term “upstream” refers to the location or position of a nucleic acid sequence relative to a reference nucleic acid sequence, particularly a position that, during RNA transcription, is closer to the 5′ end of the transcribed RNA molecule encoded by the reference sequence. For example, for two sequences, A and B, such that sequence A is upstream of sequence B, transcription of sequence B proceeds away from sequence A.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Standard techniques may be used for recombinant DNA, oligonucleotide synthesis, and tissue culture and transformation (e.g., electroporation. lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

The helper functions adenoviruses provide for AAV replication have been previously described. Without wishing to be bound by any particular hypothesis, adenoviral E1A protein has been described to activate AAV gene expression by binding and activating the AAV P5 rep promoter. Similarly. E2A. another adenoviral protein, has been described to activate AAV P5 promoter transcription. E2A has also been described to cooperate with virus associated RNA I (VA RNAI) to enhance the translation of AAV RNAs. Adenoviral E4orf4 has been shown to induce cell-cycle arrest at the G2/M border, as well as to aid in AAV production. Adenoviral E4orf6 has been described to enhance the conversion of single-stranded recombinant AAV genomes into double-stranded genomes, a rate-limiting step of viral DNA-replication both in vitro and in vivo. VA RNAI has also been described to support AAV replication. It has been described that VA RNAI physically interacts with the double-stranded RNA-activated protein kinase (PKR), which would otherwise elicit an antiviral immune response blocking viral protein production.

Prior studies have suggested that in HEK293 cells, which provide the E1 gene, the minimal set of genes in trans for efficient recombinant AAV production is E2a, E4orf6 and the VA RNAI gene. A helper plasmid named pXX6-80, containing this set of genes as well as additional genes that may not be necessary for production, is used for the production of adenovirus-free recombinant AAV.

One major ongoing challenge in the development and optimization of AAV vectors for clinical applications is to increase the amount of viruses being produced. Due to their non-proliferative nature, their production depends solely on the transfection efficiency of the parvoviral genomic components into the packaging cell lines (for example human embryonic kidney cells, HEK293 or HEK293T, or insect cells e.g., Sf9). Thus, it remains of high importance to develop means to increase recombinant AAV (rAAV) production.

Other major challenges related to the production of rAAVs for clinical applications are those related to the cost to produce such rAAVs in large quantities, and also to the safety of the final products themselves. For example, commercially available helper plasmids, such as pXX6-80, appear to transcribe low levels of the Ad fiber protein. Importantly, the fiber protein is not required for AAV production, and could be immunogenic in humans. In addition, the size of pXX6-80 is rather large, at over 18 kb. This large plasmid size increases the difficulty and cost of its manufacturing, which can be highly impactful when sourcing GMP plasmids for the manufacturing of clinical-grade AAV.

Different versions of adenoviral helper plasmids have been derived by others, including, for example, pFAdDeltaF6 (derived at the University of Pennsylvania) and pHelper (Agilent). The pFAdDeltaF6 plasmid is about 3 kb smaller than pXX6-80, but retains the fiber gene sequence. The pHelper plasmid, which is available from Agilent, is smaller than pXX6-80, at about 11.6 kb. However, it contains an ampicillin resistance gene, which is generally discouraged for plasmids used in AAV production.

The present disclosure addresses the above-described technical challenges by providing compositions and methods described herein.

In some embodiments, the present disclosure relates to adenovirus derived helper plasmids (an adenoviral helper plasmid) comprising adenoviral DNA sequences encoding viral helper proteins. In some embodiments, adenoviral helper plasmids of the present invention are used in methods of production of recombinant adeno-associated viruses (rAAVs). In some embodiments, adenoviral helper plasmids of the present disclosure increase production of rAAVs.

In some embodiments, the present disclosure provides an adenoviral helper plasmid comprising nucleotide sequences encoding proteins derived from sources that are not adenovirus. In some embodiments, the present disclosure provides an adenoviral helper plasmid comprising nucleotide sequences encoding proteins derived from viruses other than adenovirus. In some embodiments, an adenoviral helper plasmid comprises all or a portion of an adenoviral nucleotide sequence encoding adenoviral proteins E2a and E4, L4 region, as well as non-coding RNA VA RNA. In some embodiments, present disclosure describes improved adenoviral helper plasmids that are smaller than the leading commercially available adenoviral helper plasmids, and that allow for safer and less costly production of rAAVs in producer cell expression systems.

In some embodiments, the present disclosure provides an adenoviral helper plasmid that has reduced overall size relative to presently available adenoviral helper plasmids (e.g., pXX6-80 at 18.932 kbp; pALD-X80 at 18.876 kbp: pHelper at 11.635 kbp; pFAdDeltaF6 at 15.420 kbp).

In some embodiments, the present disclosure provides adenoviral helper plasmids having a smaller size. In some embodiments, an adenoviral helper plasmid of the present disclosure is approximately between 6.5 kb and 15.5 kb. In some embodiments, an adenoviral helper plasmid of the present disclosure has a size that is approximately 6 kb, 7 kb, 8 kb, 9 kb, 10 kb, 11 kb, 12 kb, 13 kb, 14 kb. 15 kb, or 16 kb. In some embodiments, an adenoviral helper plasmid of the present disclosure has a size that is approximately 6-7 kb; 6.5-7.5 kb; 7-8 kb; 7.5-8.5 kb; 8-9 kb; 8.5-9.5 kb; 9-10 kb; 9.5-10.5 kb; 10-11 kb; 10.5-11.5 kb; 11-12 kb; 11.5-12.5 kb; 12-13 kb; 12.5-13.5 kb; 13-14 kb; 13.5-14.5 kb; 14-15 kb; 14.5-15.5 kb: 15-16 kb. The smaller size of the adenoviral helper plasmids of the present disclosure enables the simpler and less costly production of AAV at the quantities necessary for large-scale manufacturing of AAV. In some embodiments, removing genes and/or portions of genes makes an adenoviral helper plasmid of the present disclosure safer, since the producing cells would not produce the adenovirus structural proteins (e.g., fiber, 100K and hexon assembly), that could co-purify with AAV during downstream processing and would therefore present a lower risk of inadvertently introducing adenovirus structural proteins to patients.

In some embodiments, removal of adenoviral helper genes resulting in a smaller adenoviral helper plasmid enables addition of supplementary genes to further improve AAV quality and yield. Although these supplementary genes increase the size of the plasmid relative to the smallest versions, they enable comparable or higher AAV productivity and are therefore worth the additional cost to produce. Importantly, these plasmids are still smaller than commercially available helper plasmids such as, for example, pALD-X80.

### Adenoviral Helper Plasmids

### Helper Genes and Resistance Genes

In some embodiments, an adenoviral helper plasmid of the present disclosure comprises one or more nucleotide sequence(s) encoding proteins selected from the group consisting of E2b, E2a, E4orf4, E1B55K, E1b19K, E1a, E4orf6, VA RNA, and combinations thereof.

In some embodiments, an adenoviral helper plasmid comprises a nucleotide sequence encoding an E4 region and a VA RNA region. In some embodiments, an E4 region comprises one or more of E4orf1, E4orf2, E4orf3, E4orf4, E4orf5, E4orf6, and E4orf7. In some embodiments. E4orf1 has a nucleotide sequence that is at least 80%, 85%, 90%, 95%. 99%, or 100% identical to SEQ ID NO: 53. In some embodiments, E4orf1 has an amino acid sequence that is at least 80%, 85%, 90%, 95%, 99%, or 100% identical to SEQ ID NO: 55. In some embodiments, E4orf2 has a nucleotide sequence that is at least 80%, 85%, 90%, 95%, 99%, or 100% identical to SEQ ID NO: 56. In some embodiments, E4orf2 has an amino acid sequence that is at least 80%, 85%, 90%, 95%, 99%, or 100% identical to SEQ ID NO: 58. In some embodiments, E4orf3 has a nucleotide sequence that is at least 80%, 85%, 90%, 95%, 99%, or 100% identical to SEQ ID NO: 59. In some embodiments, E4orf3 has an amino acid sequence that is at least 80%, 85%, 90%, 95%, 99%, or 100% identical to SEQ ID NO: 61. In some embodiments, E4orf4 has a nucleotide sequence that is at least 80%, 85%, 90%, 95%, 99%, or 100% identical to SEQ ID NO: 62. In some embodiments, E4orf4 has an amino acid sequence that is at least 80%, 85%, 90%, 95%, 99%, or 100% identical to SEQ ID NO: 64. In some embodiments, E4orf6 has a nucleotide sequence that is at least 80%, 85%, 90%, 95%, 99%, or 100% identical to SEQ ID NO: 65. In some embodiments. E4orf6 has an amino acid sequence that is at least 80%, 85%, 90%, 95%. 99%, or 100% identical to SEQ ID NO: 67. In some embodiments, E4orf7 has a nucleotide sequence that is at least 80%, 85%, 90%, 95%, 99%, or 100% identical to SEQ ID NO: 68. In some embodiments, E4orf7 has an amino acid sequence that is at least 80%, 85%, 90%, 95%, 99%, or 100% identical to SEQ ID NO: 69. In some embodiments, an adenoviral helper plasmid does not comprise a nucleotide sequence comprising E4orf1. In some embodiments, an adenoviral helper plasmid does not comprise a nucleotide sequence comprising E4orf2. In some embodiments, an adenoviral helper plasmid does not comprise a nucleotide sequence comprising E4orf1 and does not comprise a nucleotide sequence comprising E4orf2. In some embodiments, expression of the E4 region is under the control of an E4 mini promoter. In some embodiments, an E4 region is operably linked to an E4 mini promoter. In some embodiments, an E4 mini promoter has a nucleotide sequence that is at least 80%, 85%, 90%, 95%, 99%, or 100% identical to SEQ ID NO: 1. In some embodiments, an E4 region is operably linked to an SV40 promoter. In some embodiments. expression of the E4 region is under the control of an SV40 promoter. In some embodiments, an SV40 promoter has a nucleotide sequence that is at least 80%, 85%, 90%, 95%, 99%, or 100% identical to SEQ ID NO: 2.

In some embodiments, an adenoviral helper plasmid of the present invention comprises a resistance gene. In some embodiments, an adenoviral helper plasmid of the present invention comprises an ampicillin resistance gene (e.g., a nucleotide sequence encoding a protein conferring resistance to ampicillin). In some embodiments, an adenoviral helper plasmid of the present invention does not comprise an ampicillin resistance gene. In some embodiments. an adenoviral helper plasmid of the present invention comprises a kanamycin resistance gene (e.g., a nucleotide sequence encoding a protein conferring resistance to kanamycin). In some embodiments, an adenoviral helper plasmid of the present invention does not comprise a kanamycin resistance gene.

### E2a Region

In some embodiments, an adenoviral helper plasmid of the present disclosure comprises an E2a region. In some embodiments an E2a region, as used herein, comprise(s) one or more nucleotide sequences encoding an E2a protein and an engineered L4 region. In some embodiments, an adenoviral helper plasmid of the present disclosure comprises an E2a region that has been modified. In some embodiments, the E2a region of an adenoviral helper plasmid of the present disclosure comprises deletions to reduce plasmid size while maintaining production of E2a protein and AAV.

In some embodiments, the E2a region has been modified to comprise deletions while keeping JEP promoter sequence that corresponds to that identified by Jing et al., 2001. Inhibition of adenovirus cytotoxicity, replication, and E2a gene expression by adeno-associated virus. Virology, 291(1), pp. 140-151, which is incorporated herein by reference for any purpose. In some embodiments, an E2a region has been modified to comprise deletions while keeping GEP promoter sequence that corresponds to that identified by Guilfoyle et al., 1985. Two functions encoded by adenovirus early region 1A are responsible for the activation and repression of the DNA-binding protein gene. The EMBO Journal, 4(3), pp. 707-713, which is incorporated herein by reference for any purpose.

In some embodiments, the E2a region has been modified to comprise deletions while keeping a certain amount of nucleotides upstream of E2a (e.g., 1 kbp or 50 bp upstream of E2a).

In some embodiments, the E2a region has been modified to comprise truncations starting from the NotI end of adenoviral helper plasmids of the present disclosure.

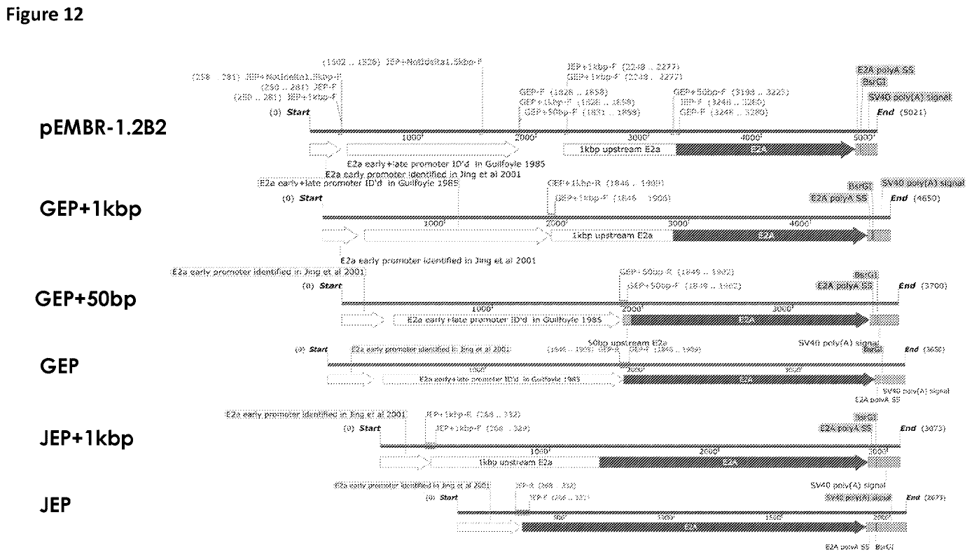

In some embodiments, the E2a region has been modified as illustrated in FIGS. 12-14 and/or 18-19 and/or 25, 29A.

### L4 Region

In some embodiments, an adenoviral helper plasmid of the present disclosure comprises an L4 region. In some embodiments, an adenoviral helper plasmid of the present disclosure comprises one or more nucleotide sequence(s) in an L4 region which encodes one or more protein(s) (e.g., 33 k, 22 k, etc.).

In some embodiments, an adenoviral helper plasmid of the present disclosure comprises an L4 region that has been modified. In some embodiments, the L4 region of an adenoviral helper plasmid of the present disclosure comprises deletions to reduce plasmid size while maintaining production of L4 33 k and L422 k protein and AAV.

In some embodiments, an adenoviral helper plasmid of the present disclosure encodes for but does not express L4 33 k and L422 k protein. In some embodiments, the L4 region of an adenoviral helper plasmid of the present disclosure comprises modifications to abolish expression of L4 33 k and L422 k protein while maintaining nucleotide sequences that encode for L4 33 k and L422 k.

In some embodiments, the L4 region of an adenoviral helper plasmid of the present disclosure comprises deletions which abolish expression of L4 100K. In some embodiments, the L4 region of an adenoviral helper plasmid of the present disclosure comprises deletions which removes nucleotides encoding L4 100K abolish expression of L4 100K.

In some embodiments, an adenoviral helper plasmid of the present disclosure comprises a nucleotide sequence encoding L4 33K. In some embodiments, an adenoviral helper plasmid of the present disclosure comprises a nucleotide sequence encoding L4 22K. In some embodiments, an adenoviral helper plasmid of the present disclosure comprises a nucleotide sequence encoding L4 33K and L4 22K. In some embodiments, an adenoviral helper plasmid of the present disclosure comprises a nucleotide sequence encoding L4 33K and L4 22K modified such that L4 33K is not expressed. In some embodiments, an adenoviral helper plasmid of the present disclosure comprises a nucleotide sequence encoding L4 33K and L4 22K modified such that L4 22 K is not expressed.

In some embodiments, an adenoviral helper plasmid of the present disclosure comprises a native L4 region (~1379 bp in length). In some embodiments, an adenoviral helper plasmid of the present disclosure comprises an L4 expression construct (~1184 bp), referred to as +L4 in adenoviral helper plasmid designs (e.g., those as described herein), which has replaced the native polyA signal with the 49 bp synthetic polyA signal (SEQ ID NO: 108). In some embodiments, an adenoviral helper plasmid of the present disclosure comprises a nucleotide sequence encoding L4 33K. In some embodiments, an adenoviral helper plasmid of the present disclosure comprises a nucleotide sequence encoding L4 22K. In some embodiments, an adenoviral helper plasmid of the present disclosure comprises a nucleotide sequence encoding L4 33K and L4 22K. In some embodiments, an adenoviral helper plasmid of the present disclosure expresses L4 genes 33K and L4 22K from a native L4 promoter. In some embodiments, an adenoviral helper plasmid of the present disclosure comprises a nucleotide sequence encoding a native L4 promoter, 50 bp nucleotides upstream of the native promoter, L4 33K and L4 22K. In some embodiments the 50 bp nucleotides upstream of the native promoter are determined relative to a reference adenovirus sequence. In some embodiments a reference sequence is GenBank: M73260.1 (Ad5 sequence).

In some embodiments, an adenoviral helper plasmid of the present disclosure does not comprise an L4 region. In some embodiments, an adenoviral helper plasmid of the present disclosure does not comprise one or more nucleotide sequence(s) in an L4 region which encode one or more protein(s) (e.g., 33 k, 22 k, etc.). In some embodiments, an adenoviral helper plasmid of the present disclosure does not comprise a nucleotide sequence encoding L4 33K. In some embodiments, an adenoviral helper plasmid of the present disclosure does not comprise a nucleotide sequence encoding L4 22K.

In some embodiments an L4 region is provided in trans relative to an adenoviral helper plasmid. In some embodiments, an L4 region is provided in a producer cell. In some embodiments, a producer cell comprises a nucleotide sequence encoding an L4 region. In some embodiments, a producer cell comprises a nucleotide sequence encoding L4 proteins L4 33K and L4 22K. In some embodiments, a producer cell comprises a nucleotide sequence encoding L4 33K. In some embodiments, a producer cell comprises a nucleotide sequence encoding L4 22K.

In some embodiments, an L4 region or portions thereof is/are provided by a plasmid other than an adenoviral helper plasmid. In some embodiments the present disclosure provides an L4 trans plasmid. In some embodiments an L4 trans plasmid comprises an adenoviral L4 region. In some embodiments, an L4 trans plasmid of the present disclosure comprises one or more nucleotide sequence(s) in an L4 region which encode one or more protein(s) (e.g., 33 k, 22 k, etc.). In some embodiments, an L4 trans plasmid of the present disclosure provides one or more nucleotide sequence(s) in an L4 region which encode one or more protein(s) (e.g., 33 k, 22 k, etc.) on a plasmid other than an adenoviral helper plasmid. As in some embodiments the one or more nucleotide sequence(s) in an L4 region which encode one or more protein(s) (e.g., 33 k, 22 k, etc.) are not provided on the same plasmid (e.g., cis) as other adenoviral helper components the plasmid is referred to as an L4 trans plasmid.

In some embodiments, an L4 trans plasmid of the present disclosure comprises one or more nucleotide sequence(s) in an L4 region which encode and express one or more protein(s) (e.g., 33 k, 22 k, etc.). In some embodiments, an L4 trans plasmid of the present disclosure comprises a nucleotide sequence that encodes for and expresses L4 33 k and L422 k protein. In some embodiments, an L4 trans plasmid of the present disclosure comprises a nucleotide sequence that encodes for and expresses L4 33 k protein. In some embodiments, an L4 trans plasmid of the present disclosure comprises a nucleotide sequence that encodes for and expresses L4 22 k protein.

In some embodiments, an L4 trans plasmid of the present disclosure comprises a nucleotide sequence encoding L4 33K. In some embodiments, an L4 trans plasmid of the present disclosure comprises a nucleotide sequence encoding L4 22K. In some embodiments, an L4 trans plasmid of the present disclosure comprises a nucleotide sequence encoding L4 33K and L4 22K. In some embodiments, an L4 trans plasmid of the present disclosure comprises a nucleotide sequence encoding L4 33K and L4 22K modified such that L4 33K is not expressed. In some embodiments, an L4 trans plasmid of the present disclosure comprises a nucleotide sequence encoding L4 33K and L4 22K modified such that L4 22 K is not expressed.

In some embodiments, expression of proteins encoded by an L4 trans plasmid of the present disclosure is under the control of a promoter. In some embodiments, expression of proteins encoded by an L4 trans plasmid of the present disclosure is under the control of an EF1alpha promoter.

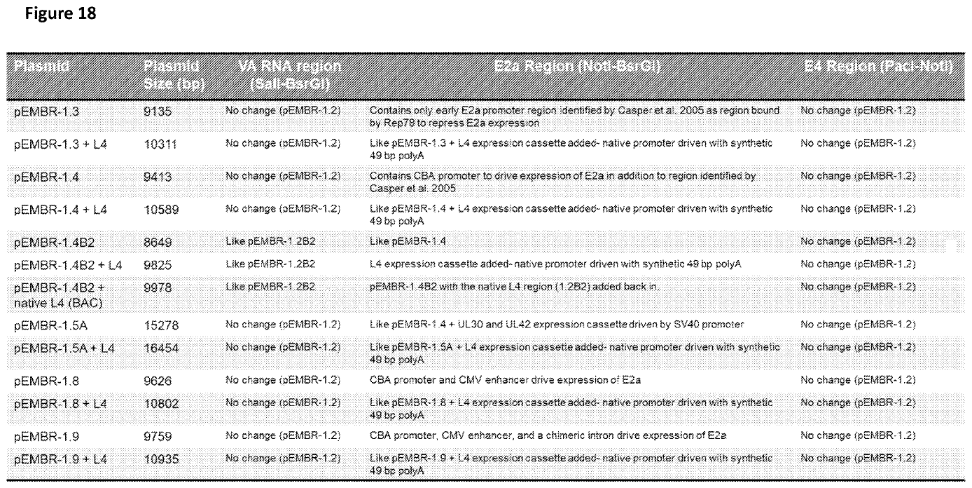

In some embodiments, an adenoviral helper plasmid of the present disclosure comprises an E2a region modified as illustrated in FIGS. 18 and/or 19 and/or 25.

### Fiber Gene

In some embodiments, an adenoviral helper plasmid of the present disclosure does not comprise a nucleotide sequence encoding adenoviral fiber protein. In some embodiments, an adenoviral helper plasmid does not comprise a nucleotide sequence encoding a full-length adenoviral fiber protein. In some embodiments, an adenoviral helper plasmid comprises a nucleotide sequence encoding a portion or fragment of adenoviral fiber protein. In some embodiments, an adenoviral helper plasmid comprises a nucleotide sequence that is at least 80%, 85%, 90%, 95%, 99%, or 100% identical to that of pXX6-80, excluding the nucleotide sequence encoding an adenoviral fiber protein.

### L1-52/55K (Packaging Protein 3) Gene

In some embodiments, an adenoviral helper plasmid of the present disclosure does not comprise a nucleotide sequence encoding a L1-52/55K (Packaging Protein 3) protein. In some embodiments, an adenoviral helper plasmid of the present invention does not comprise a nucleotide sequence encoding Peripentonal Hexon-Associated genes.

### L4 Region

In some embodiments, an adenoviral helper plasmid of the present disclosure comprises a complete L4 (hexon assembly) gene. In some embodiments, an adenoviral helper plasmid of the present disclosure comprises a nucleotide sequence encoding a complete L4 (hexon assembly). In some embodiments, an adenoviral helper plasmid of the present disclosure comprises a nucleotide sequence that is at least 80%, 85%, 90%, 95%, 99%, or 100% identical to SEQ ID NO: 3. In some embodiments, an adenoviral helper plasmid of the present disclosure comprises an amino acid sequence that is at least 80%, 85%, 90%, 95%, 99%, or 100% identical to SEQ ID NO: 4. In some embodiments, an adenoviral helper plasmid of the present invention comprises a complete L4 (33 kDa Ex2) gene. In some embodiments, an adenoviral helper plasmid of the present disclosure comprises a nucleotide sequence encoding a complete L4 (33 kDa Ex2). In some embodiments, an adenoviral helper plasmid of the present disclosure comprises a nucleotide sequence that is at least 80%, 85%, 90%, 95%, 99%, or 100% identical to SEQ ID NO: 5. In some embodiments, an adenoviral helper plasmid of the present disclosure comprises an amino acid sequence that is at least 80%, 85%, 90%, 95%. 99%, or 100% identical to SEQ ID NO: 6.

In some embodiments, an adenoviral helper plasmid of the present disclosure comprises a complete L4 Encapsidation Protein gene. In some embodiments, an adenoviral helper plasmid of the present disclosure comprises a nucleotide sequence encoding a complete L4 Encapsidation Protein. In some embodiments, an adenoviral helper plasmid of the present disclosure comprises a nucleotide sequence that is at least 80%, 85%, 90%, 95%, 99%, or 100% identical to SEQ ID NO: 7. In some embodiments, an adenoviral helper plasmid of the present disclosure comprises an amino acid sequence that is at least 80%, 85%, 90%, 95%, 99%, or 100% identical to SEQ ID NO: 8.

In some embodiments, an adenoviral helper plasmid of the present disclosure does not comprise an L4 (hexon assembly) gene. In some embodiments, an adenoviral helper plasmid does not comprise an L4 Encapsidation Protein gene. In some embodiments, an adenoviral helper plasmid does not comprise an L4 (hexon assembly) gene and does not comprise an L4 Encapsidation Protein gene. In some embodiments, an adenoviral helper plasmid of the present disclosure does not comprise a nucleotide sequence encoding L4 (hexon assembly). In some embodiments, an adenoviral helper plasmid does not comprise a nucleotide sequence encoding L4 Encapsidation Protein. In some embodiments, an adenoviral helper plasmid does not comprise a nucleotide sequence encoding L4 (hexon assembly) and does not comprise a nucleotide sequence encoding L4 Encapsidation Protein gene. In some embodiments, an adenoviral helper plasmid of the present disclosure comprises a nucleotide sequence encoding a fragment of L4 33 kDa Ex2. In some embodiments, an adenoviral helper plasmid of the present disclosure comprises a nucleotide sequence that is at least 80%, 85%, 90%, 95%, 99%, or 100% identical to SEQ ID NO: 9. In some embodiments, an adenoviral helper plasmid comprises a nucleotide sequence encoding a fragment of L4 33 kDa Ex2. In some embodiments, an adenoviral helper plasmid of the present disclosure comprises an amino acid sequence that is at least 80%, 85%, 90%, 95%, 99%, or 100% identical to SEQ ID NO: 10. In some embodiments, a nucleotide sequence encoding a fragment of L4 33 kDa Ex2 comprises an E2a promoter region (see, for example, Casper et al., “Identification of an adeno-associated virus Rep protein binding site in the adenovirus E2a promoter.” Journal of virology 79.1 (2005)). In some embodiments, an E2a promoter region has a nucleotide sequence that is at least 80%, 85%, 90%, 95%, 99%, or 100% identical to SEQ ID NO: 11. In some embodiments, an adenoviral helper plasmid does not comprise a nucleotide sequence encoding a fragment of L4 33 kDa Ex2. In some embodiments, an adenoviral helper plasmid does not comprise an E2a promoter region.

In some embodiments, an adenoviral helper plasmid of the present disclosure comprises a nucleotide sequence encoding a fragment of hexon-associated precursor (L4 pVIII). In some embodiments, an adenoviral helper plasmid comprises a nucleotide sequence that is at least 80%, 85%, 90%, 95%. 99%, or 100% identical to SEQ ID NO: 12. In some embodiments, an adenoviral helper plasmid comprises an amino acid sequence that is at least 80%, 85%, 90%, 95%, 99%, or 100% identical to SEQ ID NO: 13. In some embodiments, an adenoviral helper plasmid does not comprise a nucleotide sequence encoding hexon-associated precursor (L4 pVIII). In some embodiments. an adenoviral helper plasmid does not comprise a nucleotide sequence encoding a fragment of partial hexon-associated precursor (L4 pVIII).

### VA RNA Region

In some embodiments, an adenoviral helper plasmid of the present disclosure comprises a VA RNA region having a nucleotide sequence that is at least 80%, 85%, 90%, 95%, 99%, or 100% identical to SEQ ID NO: 14. In some embodiments, an adenoviral helper plasmid comprises a VA RNA region having a nucleotide sequence that is at least 80%, 85%, 90%, 95%, 99%, or 100% identical to SEQ ID NO: 15. In some embodiments, a VA RNA region comprises a VA RNAI gene having a sequence that is at least 80%, 85%, 90%, 95%, 99%, or 100% identical to SEQ ID NO: 16. In some embodiments, a VA RNA region comprises a VA RNAI gene having a sequence that is at least 80%, 85%, 90%, 95%, 99%, or 100% identical to SEQ ID NO: 17. In some embodiments, a VA RNA region comprises a VA RNAII gene having a sequence that is at least 80%, 85%, 90%, 95%, 99%, or 100% identical to SEQ ID NO: 18. In some embodiments, a VA RNA region comprises a VA RNAII gene having a sequence that is at least 80%, 85%, 90%, 95%, 99%, or 100% identical to SEQ ID NO: 19.

In some embodiments, an adenoviral helper plasmid of the present disclosure comprises a nucleotide sequence encoding a fragment of DNA Terminal Protein. In some embodiments, a nucleotide sequence encoding a fragment of DNA Terminal Protein is at least 80%, 85%, 90%, 95%, 99%, or 100% identical to SEQ ID NO: 20. In some embodiments, a fragment of DNA Terminal Protein has an amino acid sequence that is at least 80%, 85%, 90%, 95%, 99%, or 100% identical to SEQ ID NO: 21. In some embodiments, an adenoviral helper plasmid does not comprise a nucleotide sequence encoding DNA Terminal Protein. In some embodiments, an adenoviral helper plasmid comprises a nucleotide sequence encoding a fragment of 23 kDa endoprotease. In some embodiments, an adenoviral helper plasmid comprise a nucleotide sequence at least 80%, 85%, 90%, 95%, 99%, or 100% identical to SEQ ID NO: 22. In some embodiments, a fragment of 23 kDa endoprotease region has an amino acid sequence that is at least 80%, 85%, 90%, 95%, 99%, or 100% identical to SEQ ID NO: 23. In some embodiments, an adenoviral helper plasmid does not comprise a nucleotide sequence encoding 23 kDa endoprotease region.

### Introduction of Genes Encoding Supplementary Features

In some embodiments, an adenoviral helper plasmid of the present disclosure comprises an E2a gene. In some embodiments, an adenoviral helper plasmid of the present disclosure comprises a nucleotide sequence encoding E2a. In some embodiments, an adenoviral helper plasmid of the present disclosure comprises a nucleotide sequence that is at least 80%, 85%, 90%, 95%, 99%, or 100% identical to SEQ ID NO: 24. In some embodiments, an adenoviral helper plasmid of the present disclosure comprises an amino acid sequence that is at least 80%, 85%, 90%, 95%, 99%, or 100% identical to SEQ ID NO: 25. In some embodiments, expression of E2a is under the control of a promoter. In some embodiments, a nucleotide sequence encoding E2a is operably linked to a promoter. In some embodiments, a promoter is, for example, a CMV promoter, a PGK promoter, an SV40 promoter, an EF-1α promoter, a Ubc promoter. a CAG promoter, or a β-actin promoter. In some embodiments, a nucleotide sequence encoding E2a is operably linked to a transcriptional enhancer. In some embodiments, a transcriptional enhancer is, for example, a CMV enhancer. In some embodiments, a nucleotide sequence encoding E2a is operably linked to a regulatory intron. In some embodiments, expression of E2a is under the control of a chicken β-actin promoter. In some embodiments, a nucleotide sequence encoding E2a is operably linked to a chicken β-actin promoter. In some embodiments, a chicken β-actin promoter has a nucleotide sequence that is at least 80%, 85%, 90%, 95%, 99%, or 100% identical to SEQ ID NO: 26. In some embodiments, a chicken β-actin promoter is positioned upstream of a nucleotide sequence encoding E2a. In some embodiments, expression of E2a is under the control of an E2a promoter and a chicken β-actin promoter. In some embodiments, a nucleotide sequence encoding E2a is operably linked to an E2a promoter and a chicken β-actin promoter. In some embodiments, a chicken β-actin promoter is positioned upstream of an E2a promoter. In some embodiments, expression of E2a is under the control of chicken β-actin promoter and a CMV enhancer. In some embodiments, a nucleotide sequence encoding E2a is operably linked to a chicken β-actin promoter and a CMV enhancer. In some embodiments, a chicken β-actin promoter and a CMV enhancer are positioned upstream of an E2a promoter. In some embodiments, an adenoviral helper plasmid comprises an E2a polyadenylation signal. In some embodiments, an E2a polyadenylation signal is positioned downstream of a nucleotide sequence encoding E2a. In some embodiments, an E2a polyadenylation signal has a nucleotide sequence that is at least 80%, 85%, 90%, 95%, 99%, or 100% identical to SEQ ID NO: 27. In some embodiments, an adenoviral helper plasmid comprises an SV40 polyadenylation signal. In some embodiments, an SV40 polyadenylation signal is positioned downstream of a nucleotide sequence encoding E2a. In some embodiments, an SV40 polyadenylation signal is positioned downstream of an E2a polyadenylation signal. In some embodiments, an SV40 polyadenylation signal has a sequence that is at least 80%, 85%, 90%, 95%, 99%, or 100% identical to SEQ ID NO: 28.

In some embodiments, an adenoviral helper plasmid comprises a nucleotide sequence encoding UL30 derived from HSV-1. In some embodiments, a nucleotide sequence encoding UL30 has a nucleotide sequence that is at least 80%, 85%, 90%, 95%, 99%, or 100% identical to SEQ ID NO: 29. In some embodiments, an amino acid sequence UL30 is at least 80%, 85%, 90%, 95%. 99%, or 100% identical to SEQ ID NO: 30. In some embodiments, an adenoviral helper plasmid comprises a nucleotide sequence encoding UL42 derived from HSV-1. In some embodiments, a nucleotide sequence encoding UL42 has a nucleotide sequence that is at least 80%, 85%, 90%, 95%, 99%, or 100% identical to SEQ ID NO: 31. In some embodiments, an amino acid sequence of UL42 is at least 80%, 85%, 90%, 95%, 99%, or 100% identical to SEQ ID NO: 32. In some embodiments, an adenoviral helper plasmid comprises a nucleotide sequence encoding UL30 derived from HSV-1, and a nucleotide sequence encoding UL42 derived from HSV-1. In some embodiments, a nucleotide sequence encoding UL30 and a nucleotide sequence encoding UL42 are separated by a P2a cleavage site. In some embodiments, a P2a cleavage site has a nucleotide sequence that is at least 80%, 85%, 90%, 95%, 99%, or 100% identical to SEQ ID NO: 33. In some embodiments, a P2a cleavage site has an amino acid sequence that is at least 80%, 85%, 90%, 95%, 99%, or 100% identical to SEQ ID NO: 34. In some embodiments, expression of UL30 and/or UL42 gene is/are under the control of an EF-1α promoter. In some embodiments, a nucleotide sequence encoding UL30 is operably linked to a promoter. In some embodiments, a nucleotide sequence encoding UL30 is operably linked to a CMV promoter, a PGK promoter, an SV40 promoter, an EF-1α promoter, a Ubc promoter, a CAG promoter, or a β-actin promoter. In some embodiments, a nucleotide sequence encoding UL30 is operably linked to a transcriptional enhancer. In some embodiments, a transcriptional enhancer is, for example, a CMV enhancer. In some embodiments, a nucleotide sequence encoding UL30 is operably linked to a regulatory intron. In some embodiments a nucleotide sequence encoding UL42 and/or a nucleotide sequence encoding UL30 are operably linked to an EF-1α promoter. In some embodiments. an EF-1α promoter has a nucleotide sequence that is at least 80%, 85%, 90%, 95%, 99%, or 100% identical to SEQ ID NO: 35. In some embodiments, expression of UL30 and/or UL42 is/are under the control of an SV40 promoter. In some embodiments, a nucleotide sequence encoding UL42 and/or a nucleotide sequence encoding UL30 are operably linked to an SV40 promoter. In some embodiments, an SV40 promoter has a nucleotide sequence that is at least 80%, 85%, 90%, 95%, 99%, or 100% identical to SEQ ID NO: 51.

In some embodiments, an adenoviral helper plasmid comprises a polyadenylation signal. In some embodiments, a polyadenylation signal is synthetic. In some embodiments, a synthetic polyadenylation signal is a 49 bp synthetic polyadenylation signal (SEQ ID NO. 108).

In some embodiments, a polyadenylation signal is a β-globin polyadenylation signal, SV40 polyadenylation signal, or a Bovine Growth Hormone (bGH) polyadenylation signal. In some embodiments, an adenoviral helper plasmid comprises a polyadenylation signal downstream of a nucleotide sequence encoding UL42. In some embodiments, an adenoviral helper plasmid comprises a β-globin polyadenylation signal downstream of a nucleotide sequence encoding UL42. In some embodiments, a β-globin polyadenylation signal has a sequence that is at least 80%, 85%, 90%, 95%, 99%, or 100% identical to SEQ ID NO: 36. In some embodiments, an adenoviral helper plasmid comprises a Bovine Growth Hormone (bGH) polyadenylation signal downstream of a nucleotide sequence encoding UL42. In some embodiments, a bGH polyadenylation signal has a sequence that is at least 80%, 85%, 90%, 95%, 99%, or 100% identical to SEQ ID NO: 52.

### Exemplary Adenoviral Helper Plasmids

In some embodiments, an adenoviral helper plasmid of the present disclosure has a nucleotide sequence that is at least 80%, 85%, 90%, 95%, 99%, or 100% identical to SEQ ID NO: 41. In some embodiments, an adenoviral helper plasmid of the present disclosure comprises the following components having nucleotide sequences are at least 80%, 85%, 90%, 95%, 99%, or 100% identical to the indicated sequences: E4 mini promoter (SEQ ID NO: 1), L4 (Hexon Assembly) (SEQ ID NO: 3; SEQ ID NO: 4), L4 (33 kDa Ex2) (SEQ ID NO: 5; SEQ ID NO: 6), L4 Encapsidation Protein (22 kDa) (SEQ ID NO: 7; SEQ ID NO: 8), L4 pVIII Hexon-Associated Precursor (SEQ ID NO: 12; SEQ ID NO: 13), VA RNA region A (SEQ ID NO: 14), VA RNAI-A (SEQ ID NO: 16), VA RNAII-A (SEQ ID NO: 18), partial DNA Terminal Protein (SEQ ID NO: 20; SEQ ID NO: 21), 23 kDa endoprotease fragment region (SEQ ID NO: 22; SEQ ID NO: 23), and E2a (SEQ ID NO: 24; SEQ ID NO: 25), and does not comprise the following components: a fiber gene, an L1-52/55K (Packaging Protein 3) gene, and Peripentonal Hexon-Associated genes.

In some embodiments, an adenoviral helper plasmid of the present disclosure has a nucleotide sequence that is at least 80%, 85%, 90%, 95%, 99%, or 100% identical to SEQ ID NO: 42. In some embodiments, an adenoviral helper plasmid of the present disclosure comprises the following components having nucleotide sequences that are at least 80%, 85%, 90%, 95%, 99%, or 100% identical to the indicated sequences: E4 mini promoter (SEQ ID NO: 1), L4 (33 kDa Ex2) (SEQ ID NO: 9: SEQ ID NO: 10), VA RNA region A (SEQ ID NO: 14), VA RNAI-A (SEQ ID NO: 16). VA RNAII-A (SEQ ID NO: 18), partial DNA Terminal Protein (SEQ ID NO: 20; SEQ ID NO: 21), 23 kDa endoprotease fragment region (SEQ ID NO: 22; SEQ ID NO: 23), and E2a (SEQ ID NO: 24; SEQ ID NO: 25), and does not comprise or encode the following components: a fiber gene, an L1-52/55K (Packaging Protein 3) gene. Peripentonal Hexon-Associated genes, full-length L4 (Hexon Assembly) gene, L4 Encapsidation Protein, and L4 pVIII Hexon-Associated Precursor.

In some embodiments, an adenoviral helper plasmid of the present disclosure has a nucleotide sequence that is at least 80%, 85%, 90%, 95%, 99%, or 100% identical to SEQ ID NO: 43. In some embodiments, an adenoviral helper plasmid of the present disclosure comprises the following components having nucleotide sequences that are at least 80%, 85%, 90%, 95%, 99%, or 100% identical to the indicated sequences: E4 mini promoter (SEQ ID NO: 1), L4 (33 kDa Ex2) (SEQ ID NO: 9: SEQ ID NO: 10), VA RNA region B (SEQ ID NO: 15), VA RNAI-B (SEQ ID NO: 17). VA RNAII-B (SEQ ID NO: 19), and E2a (SEQ ID NO: 24; SEQ ID NO: 25), and does not comprise or encode the following components: a fiber gene, an L1-52/55K (Packaging Protein 3) gene, Peripentonal Hexon-Associated genes, full-length L4 (Hexon Assembly) gene, L4 Encapsidation Protein, L4 pVIII Hexon-Associated Precursor. DNA Terminal Protein, and 23 kDa endoprotease fragment region.

In some embodiments, an adenoviral helper plasmid of the present disclosure has a nucleotide sequence that is at least 80%, 85%, 90%, 95%, 99%, or 100% identical to SEQ ID NO: 44. In some embodiments, an adenoviral helper plasmid of the present disclosure comprises the following components having nucleotide sequences that are at least 80%, 85%, 90%, 95%, 99%, or 100% identical to the indicated sequences: E4 mini promoter (SEQ ID NO: 1), L4 (33 kDa Ex2) (SEQ ID NO: 9; SEQ ID NO: 10), VA RNA region A (SEQ ID NO: 14), VA RNAI-A (SEQ ID NO: 16), VA RNAII-A (SEQ ID NO: 18), partial DNA Terminal Protein (SEQ ID NO: 20; SEQ ID NO: 21), 23 kDa endoprotease fragment region (SEQ ID NO: 22; SEQ ID NO: 23), E2a (SEQ ID NO: 24; SEQ ID NO: 25). and a chicken β-actin promoter upstream of E2a, and does not comprise the following components: a fiber gene, an L1-52/55K (Packaging Protein 3) gene, Peripentonal Hexon-Associated genes, full-length L4 (Hexon Assembly) gene, L4 Encapsidation Protein, and L4 pVIII Hexon-Associated Precursor.

In some embodiments, an adenoviral helper plasmid of the present disclosure has a nucleotide sequence that is at least 80%, 85%, 90%, 95%, 99%, or 100% identical to SEQ ID NO: 45. In some embodiments, an adenoviral helper plasmid of the present disclosure comprises the following components having nucleotide sequences that are at least 80%, 85%, 90%, 95%, 99%, or 100% identical to the indicated sequences: E4 mini promoter (SEQ ID NO: 1), L4 (33 kDa Ex2) (SEQ ID NO: 9; SEQ ID NO: 10), VA RNA region B (SEQ ID NO: 15), VA RNAI-B (SEQ ID NO: 17), VA RNAII-B (SEQ ID NO: 19), E2a (SEQ ID NO: 24; SEQ ID NO: 25), and a chicken β-actin promoter upstream of E2a, and does not comprise or encode the following components: a fiber gene, an L1-52/55K (Packaging Protein 3) gene, Peripentonal Hexon-Associated genes, full-length L4 (Hexon Assembly) gene, L4 Encapsidation Protein, L4 pVIII Hexon-Associated Precursor, DNA Terminal Protein, and 23 kDa endoprotease fragment region.

In some embodiments, an adenoviral helper plasmid of the present disclosure has a nucleotide sequence that is at least 80%, 85%, 90%, 95%, 99%, or 100% identical to SEQ ID NO: 46. In some embodiments, an adenoviral helper plasmid of the present disclosure comprises the following components having nucleotide sequences that are at least 80%, 85%, 90%, 95%, 99%, or 100% identical to the indicated sequences: E4 mini promoter (SEQ ID NO: 1), L4 (33 kDa Ex2) (SEQ ID NO: 9; SEQ ID NO: 10), VA RNA region B (SEQ ID NO: 15), VA RNAI-B (SEQ ID NO: 17), VA RNAII-B (SEQ ID NO: 19), E2a (SEQ ID NO: 24; SEQ ID NO: 25), SV40 polyadenylation signal downstream of E2a (SEQ ID NO: 28). and a chicken β-actin promoter upstream of E2a, and does not comprise or encode the following components: a fiber gene, an L1-52/55K (Packaging Protein 3) gene, Peripentonal Hexon-Associated genes, full-length L4 (Hexon Assembly) gene, L4 Encapsidation Protein, L4 pVIII Hexon-Associated Precursor, DNA Terminal Protein, and 23 kDa endoprotease fragment region.

In some embodiments, an adenoviral helper plasmid of the present disclosure has a nucleotide sequence that is at least 80%, 85%, 90%, 95%, 99%, or 100% identical to SEQ ID NO: 47. In some embodiments, an adenoviral helper plasmid of the present disclosure comprises the following components having nucleotide sequences that are at least 80%, 85%, 90%, 95%, 99%, or 100% identical to the indicated sequences: E4 mini promoter (SEQ ID NO: 1), L4 (33 kDa Ex2) (SEQ ID NO: 9; SEQ ID NO: 10), VA RNA region A (SEQ ID NO: 14), VA RNAI-A (SEQ ID NO: 16), VA RNAII-A (SEQ ID NO: 18), partial DNA Terminal Protein (SEQ ID NO: 20: SEQ ID NO: 21), 23 kDa endoprotease fragment region (SEQ ID NO: 22; SEQ ID NO: 23). E2a (SEQ ID NO: 24; SEQ ID NO: 25). a chicken β-actin promoter upstream of E2a, an HSV-1-derived UL30 gene (SEQ ID NO: 29; SEQ ID NO: 30), an HSV-1-derived UL42 gene (SEQ ID NO: 31; SEQ ID NO: 32), EF-1α promoter (SEQ ID NO: 35) upstream of UL30. and a R-globin polyadenylation signal (SEQ ID NO: 36) downstream of UL42, and does not comprise or encode the following components: a fiber gene, an L1-52/55K (Packaging Protein 3) gene, Peripentonal Hexon-Associated genes, full-length L4 (Hexon Assembly) gene, L4 Encapsidation Protein, and L4 pVIII Hexon-Associated Precursor.

In some embodiments, an adenoviral helper plasmid of the present disclosure has a nucleotide sequence that is at least 80%, 85%, 90%, 95%, 99%, or 100% identical to SEQ ID NO: 48. In some embodiments, an adenoviral helper plasmid of the present disclosure comprises the following components having nucleotide sequences that are at least 80%, 85%, 90%, 95%, 99%, or 100% identical to the indicated sequences: E4 mini promoter (SEQ ID NO: 1), L4 (33 kDa Ex2) (SEQ ID NO: 9; SEQ ID NO: 10), VA RNA region A (SEQ ID NO: 14), VA RNAI-A (SEQ ID NO: 16), VA RNAII-A (SEQ ID NO: 18), partial DNA Terminal Protein (SEQ ID NO: 20; SEQ ID NO: 21), 23 kDa endoprotease fragment region (SEQ ID NO: 22; SEQ ID NO: 23), E2a (SEQ ID NO: 24; SEQ ID NO: 25), a chicken β-actin promoter upstream of E2a, an HSV-1-derived UL30 gene (SEQ ID NO: 29; SEQ ID NO: 30), an HSV-1-derived UL42 gene (SEQ ID NO: 31; SEQ ID NO: 32), SV40 promoter (SEQ ID NO: 51) upstream of UL30, and a Bovine Growth Hormone (bGH) polyadenylation signal (SEQ ID NO: 52) downstream of UL42, and does not comprise or encode the following components: a fiber gene, an L1-52/55K (Packaging Protein 3) gene, Peripentonal Hexon-Associated genes, full-length L4 (Hexon Assembly) gene, L4 Encapsidation Protein, and L4 pVIII Hexon-Associated Precursor.

In some embodiments, an adenoviral helper plasmid of the present disclosure has a nucleotide sequence that is at least 80%, 85%, 90%, 95%, 99%, or 100% identical to SEQ ID NO: 49. In some embodiments, an adenoviral helper plasmid of the present disclosure comprises the following components having nucleotide sequences that are at least 80%, 85%, 90%, 95%, 99%, or 100% identical to the indicated sequences: SV40 promoter upstream of E4 region (SEQ ID NO: 2), VA RNA region B (SEQ ID NO: 15), VA RNAI-B (SEQ ID NO: 17), VA RNAII-B (SEQ ID NO: 19), E2a (SEQ ID NO: 24; SEQ ID NO: 25), SV40 polyadenylation signal downstream of E2a (SEQ ID NO: 28), SV40 polyadenylation signal downstream of E4orf6 (SEQ ID NO: 50), and a chicken β-actin promoter upstream of E2a. and does not comprise or encode the following components: a fiber gene, an L1-52/55K (Packaging Protein 3) gene, Peripentonal Hexon-Associated genes, full-length L4 (Hexon Assembly) gene, L4 Encapsidation Protein, L4 pVIII Hexon-Associated Precursor, L4 (33 kDa Ex2), DNA Terminal Protein, and 23 kDa endoprotease fragment region, E4 mini promoter upstream of E4 region, a gene encoding E4orf1, a gene encoding E4orf2, and a gene encoding E4orf3.

In some embodiments, an adenoviral helper plasmid of the present disclosure has a nucleotide sequence that is at least 80%, 85%, 90%, 95%, 99%, or 100% identical to SEQ ID NO: 94. In some embodiments, an adenoviral helper plasmid is designed to include “B2” designs comprising a SV40 polyA site to potentially increase expression of E2A and a synthesized sequence of a smaller VA region (contains Ad2 VA RNA I and VA RNA II) that does not contain the flanking Ad Terminal Protein nor Endoprotease gene sequences. In some embodiments, this region was synthesized with flanking StuI and BsrGI sites and the insert was cloned into pEMBR-1.2 to make pEMBR-1.2B2 (e.g., SEQ ID NO: 94).]

In some embodiments, adenoviral helper plasmids as described herein are 11000 bp or fewer. In some embodiments, adenoviral helper plasmids as described herein are 10000 bp or fewer. In some embodiments, adenoviral helper plasmids as described herein are 9500 bp or fewer. In some embodiments, adenoviral helper plasmids as described herein are 9000 bp or fewer, In some embodiments, adenoviral helper plasmids as described herein are 8000 bp or fewer. In some embodiments, adenoviral helper plasmids as described herein are 7500 bp or fewer. In some embodiments, adenoviral helper plasmids as described herein are 7000 bp or fewer.

In some embodiments, an L4 trans plasmid comprises a nucleotide sequence that is at least 80%, 85%, 90%, 95%, 99%, or 100% identical to SEQ ID NO: 115. In some embodiments, an L4 trans plasmid comprises a nucleotide sequence that is at least 80%, 85%, 90%, 95%, 99%, or 100% identical to SEQ ID NO: 119. In some embodiments, an L4 trans plasmid comprises a nucleotide sequence that is at least 80%, 85%, 90%, 95%, 99%, or 100% identical to SEQ ID NO: 8.

### Methods of Production

In some embodiments, an adenoviral helper plasmid of the present disclosure is useful in methods of producing rAAV. In some embodiments, an adenoviral helper plasmid and an L4 trans plasmid of the present disclosure are useful in methods of producing rAAV. In some embodiments. rAAV is produced by transfection of a producer cell. In some embodiments, a producer cell is a mammalian cell. In some embodiments, a producer cell is a transformed mammalian cell. In some embodiments, a producer cell is a Vero, HeLa, HEK293, HEK293T cell or derivative thereof.

In some embodiments, an L4 region is provided in a producer cell. In some embodiments, a producer cell comprises a nucleotide sequence encoding an L4 region. In some embodiments, a producer cell comprises a nucleotide sequence encoding L4 proteins L4 33K and L4 22K. In some embodiments, a producer cell comprises a nucleotide sequence encoding L4 33K. In some embodiments, a producer cell comprises a nucleotide sequence encoding L4 22K. In some embodiments, a method of producing a rAAV comprises transfection of a producer cell with an AAV vector plasmid, an AAV Rep-Cap expressing plasmid. and an adenoviral helper plasmid. In some embodiments, a method of producing a rAAV comprises transfection of a producer cell with an AAV vector plasmid, an AAV Rep-Cap expressing plasmid, an adenoviral helper plasmid, and an L4 trans plasmid. In some embodiments, a method of producing a rAAV comprises transfection of a producer cell with an AAV vector plasmid. an AAV Rep-Cap expressing plasmid, an adenoviral helper plasmid comprising an L4 region, and an L4 trans plasmid. In some embodiments, a method of producing a rAAV comprises transfection of a producer cell with an AAV vector plasmid, an AAV Rep-Cap expressing plasmid, an adenoviral helper plasmid comprising an L4 region, and an L4 trans plasmid comprising a nucleotide sequence encoding L4 22K. In some embodiments, a method of producing a rAAV comprises transfection of a producer cell with an AAV vector plasmid, an AAV Rep-Cap expressing plasmid, an adenoviral helper plasmid comprising an L4 region, and an L4 trans plasmid comprising a nucleotide sequence encoding L4 33K. In some embodiments, a method of producing a rAAV comprises transfection of a producer cell with an AAV vector plasmid, an AAV Rep-Cap expressing plasmid, and an adenoviral helper plasmid comprising an L4 region, wherein the producer cell comprises a nucleotide sequence encoding an L4 region or portions thereof (e.g., L33K and/or L22K).

In some embodiments, an AAV vector plasmid comprises AAV inverted terminal repeats (ITRs) and a transgene of interest. In some embodiments, an adenoviral helper plasmid is any adenoviral helper plasmid described herein. In some embodiments, an L4 trans plasmid is any L4 trans plasmid as described herein.

In some embodiments, a method of producing a rAAV comprises transfection of a producer cell stably expressing Rep-Cap. In some embodiments, a method of producing a rAAV comprises transfection of a producer cell stably expressing Rep-Cap with an AAV vector plasmid and an adenoviral helper plasmid. In some embodiments, a method of producing a rAAV comprises transfection of a producer cell stably expressing Rep-Cap with an AAV vector plasmid, an adenoviral helper plasmid and an L4 trans plasmid. In some embodiments, a method of producing a rAAV comprises transfection of a producer cell stably expressing Rep-Cap with an AAV vector plasmid, an adenoviral helper plasmid comprising an L4 region, and an L4 trans plasmid. In some embodiments, a method of producing a rAAV comprises transfection of a producer cell stably expressing Rep-Cap with an AAV vector plasmid, an adenoviral helper plasmid comprising an L4 region, and an L4 trans plasmid comprising a nucleotide sequence encoding L4 22K. In some embodiments, a method of producing a rAAV comprises transfection of a producer cell stably expressing Rep-Cap with an AAV vector plasmid, an adenoviral helper plasmid comprising an L4 region, and an L4 trans plasmid comprising a nucleotide sequence encoding L4 33K. In some embodiments, a method of producing a rAAV comprises transfection of a producer cell stably expressing Rep-Cap and comprising a nucleotide sequence encoding an L4 region or portions thereof (e.g., L33K and/or L22K) with an AAV vector plasmid, an AAV Rep-Cap expressing plasmid. and an adenoviral helper plasmid comprising an L4 region.

In some embodiments, an AAV vector plasmid comprises AAV inverted terminal repeats (ITRs) and a transgene of interest. In some embodiments, an adenoviral helper plasmid is any adenoviral helper plasmid described herein. In some embodiments, an L4 trans plasmid is any L4 trans plasmid as described herein.

### EXEMPLIFICATION

The main purpose of the work described in this disclosure is to develop novel adenoviral helper plasmids for rAAV production that are smaller, contain fewer non-necessary adenoviral genes, and that function as well or better than the most commonly used adenoviral helper plasmids.

The plasmids provided in this disclosure were synthesized de novo, were sequence-verified, and were scaled up for use in large-scale rAAV manufacturing. Production of rAAV studies were performed to compare vector yields when using the provided plasmids versus other commercially available adenoviral helper plasmids. Vector quality and activity were also assessed from rAAV produced with the different adenoviral helper plasmids to confirm that rAAV produced with the provided plasmids is at least equivalent, if not superior in quality. Taken together, these following examples demonstrate that provided adenoviral helper plasmids generate rAAV of high yield and quality, in a potentially safer and more cost-effective design.

### Example 1: Exemplary Methods for the Production of rAAVs Using Adenoviral Helper Plasmids Described Herein

HEK293 cells were transfected with a control adenoviral helper plasmid (e.g., a commercially available plasmid, such as pALD-X80, or an adenoviral helper plasmid described in herein). The adenoviral helper plasmid was co-transfected along with pAAVrep2cap9 and pAAV-CMV-GFP or pAAV-CAG-GFP plasmids using PEI transfection in order to generate AAV9/GFP. Four days post-transfection, the HEK293 cells were harvested via 0.5% Triton X-100 lysis and nuclease addition (to degrade RNA, cellular genomic DNA, and remaining plasmid DNA). After 3-4 hours of lysis/nuclease treatment, the cell lysate was sampled and submitted for qPCR titer analysis. Samples were treated with another nuclease, then EDTA and heat-treated, followed by qPCR of diluted samples to determine vector genome copy number per sample. As a metric of transfection efficiency, cells positive for GFP were quantified using fluorescence microscopy.

### Example 2: Adenoviral Helper Plasmid Lacking Fiber, L1-52/55K, and Peripentonal Hexon-Associated Genes, and Having a Partial L4 Hexon-Associated Precursor

To reduce the size of the adenoviral helper plasmid, an adenoviral helper plasmid (pEMBR-1.2: SEQ ID NO: 41) was designed which lacks the fiber gene, the L1-52/55K (Packaging Protein 3) gene, and most of the Hexon Associated Precursor, as well as the Peripentonal Hexon-Associated protein. These deletions were made relative to commercially available helper plasmids, such as pXX6-80. The Adenoviral helper genes were synthesized and assembled into a kanamycin-resistant plasmid backbone. The resulting plasmid is approximately 6.7 kb smaller than pXX6-80.

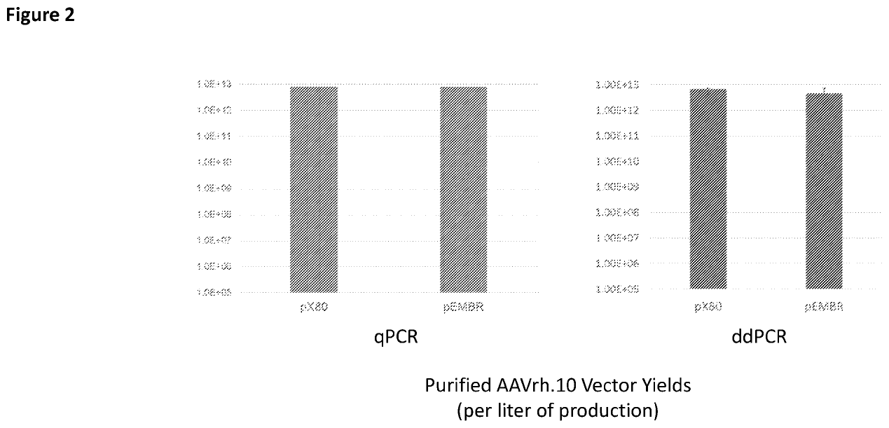

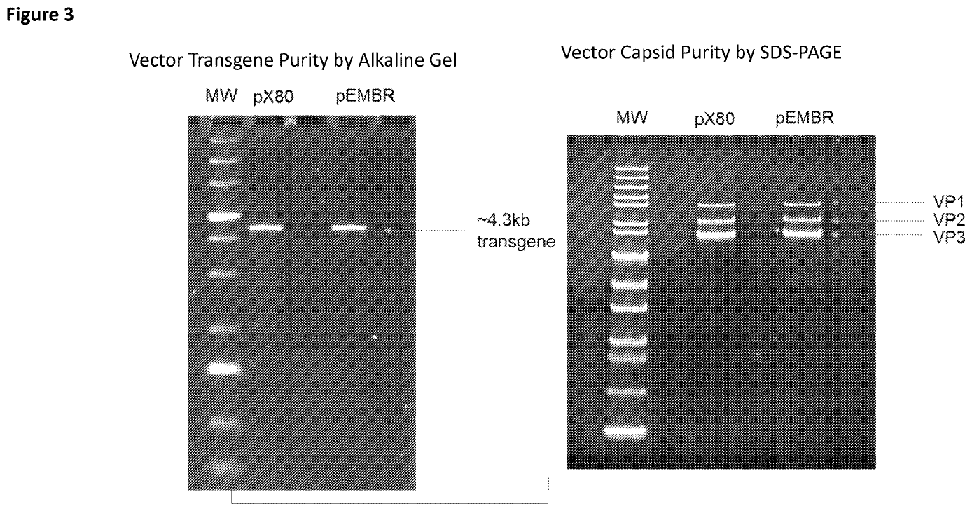

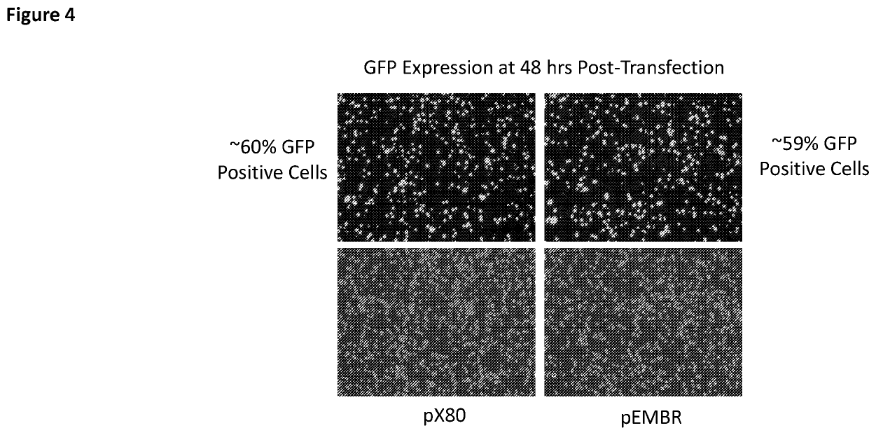

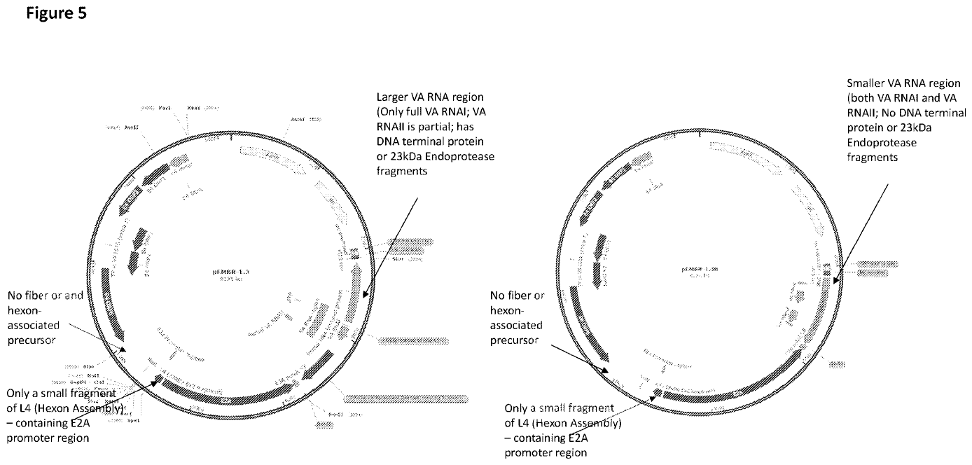

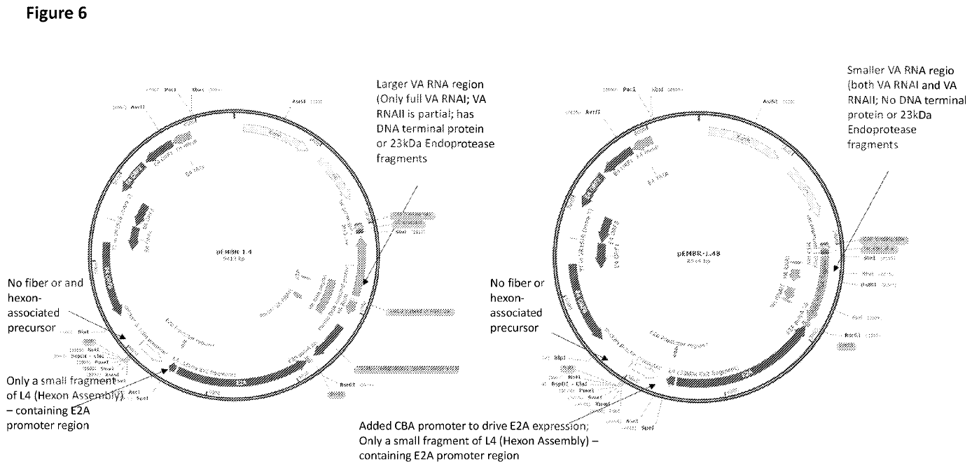

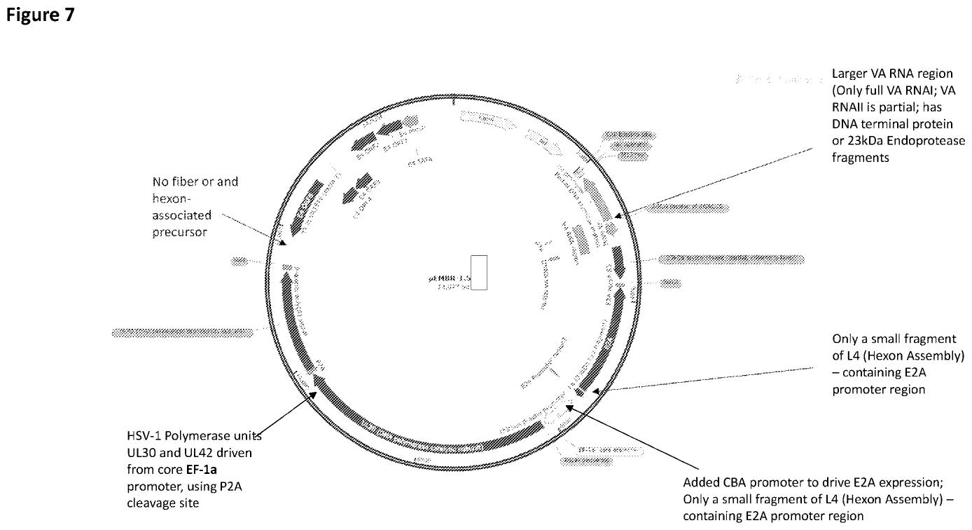

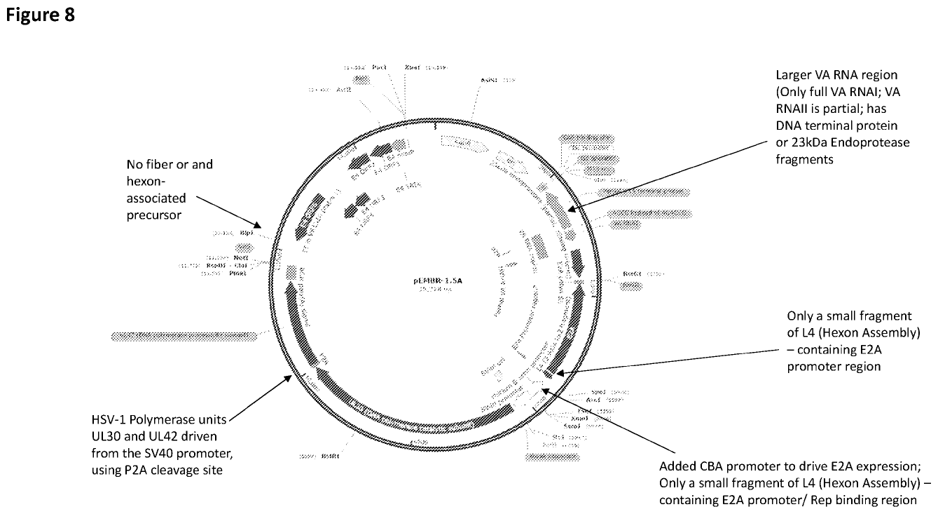

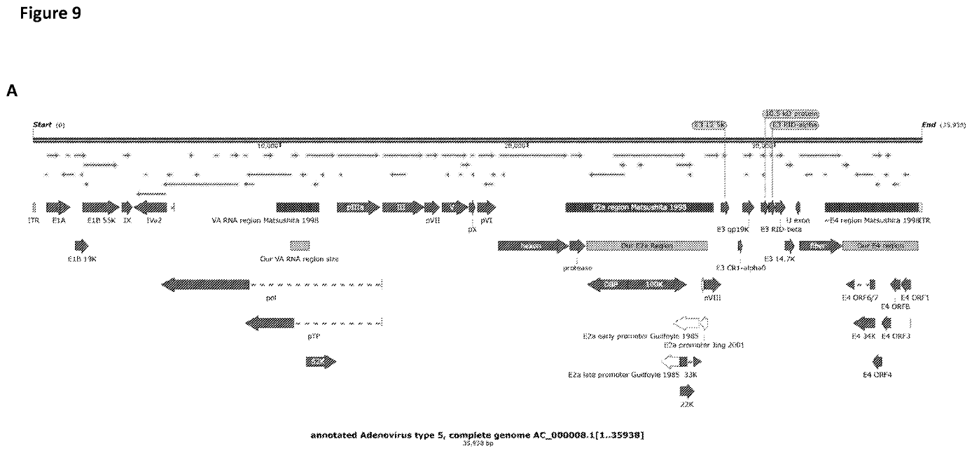

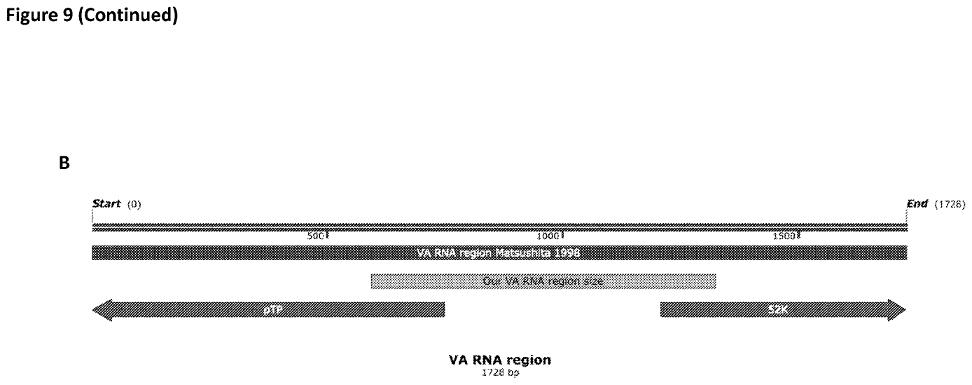

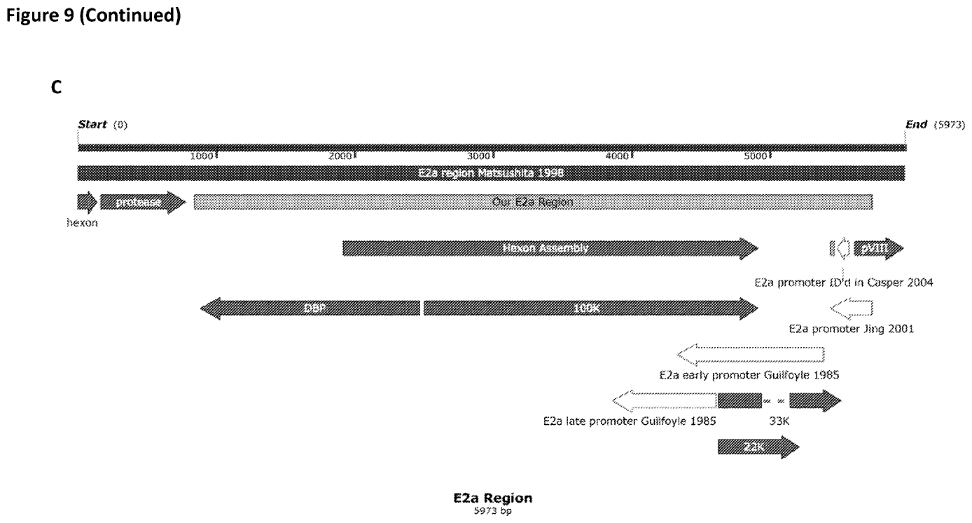

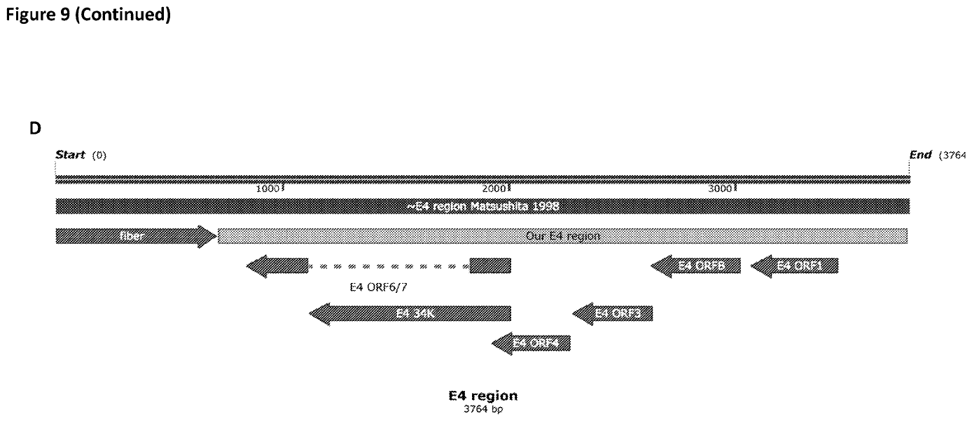

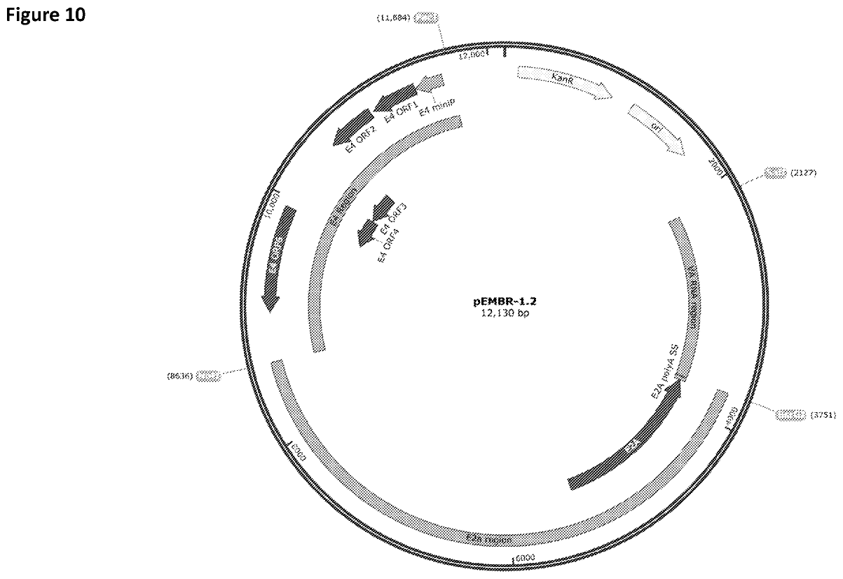

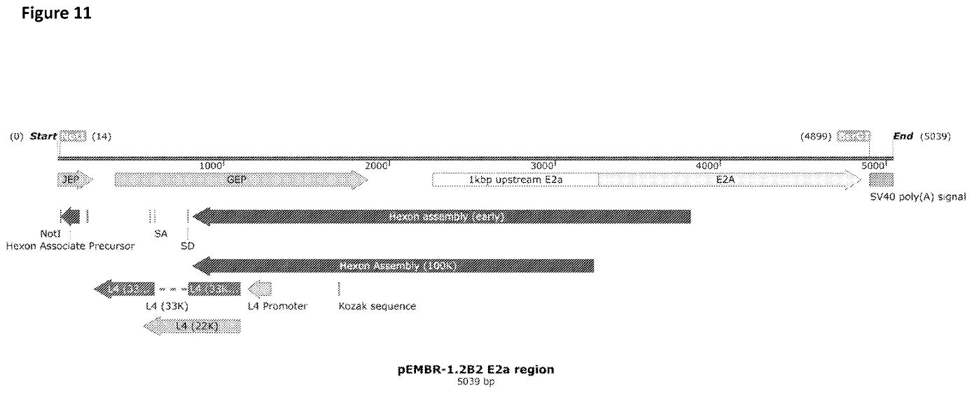

The adenoviral helper plasmid described above enabled the production of AAV in HEK293 cells. No major difference in AAV vector yield was observed between cells transfected with pALD-X80, and cells transfected with pEMBR-1.2 as measured by qPCR (See FIG. 2). rAAV vector produced with pEMBR-1.2 produces anormal vector with the correct ratios of the VP proteins, as observed when assessing vector capsid purity by SDS-PAGE (See FIG. 3), and the correct size of packaged transgene, as observed when assessing vector transgene purity by alkaline gel electrophoresis (See FIG. 3). Further. pEMBR-1.2 enabled the production of a fully functional vector capable of transfecting cells. No difference was observed in the transfection of HEK293 cells to generate AAVRH.10/ssCMV-GFP produced with pALD-X80 or pEMBR-1.2 (See FIG. 4).

### Example 3: Modifications to the E2a Region of Adenoviral Helper Plasmids to Reduce Plasmid Size while Maintaining Production of AAV

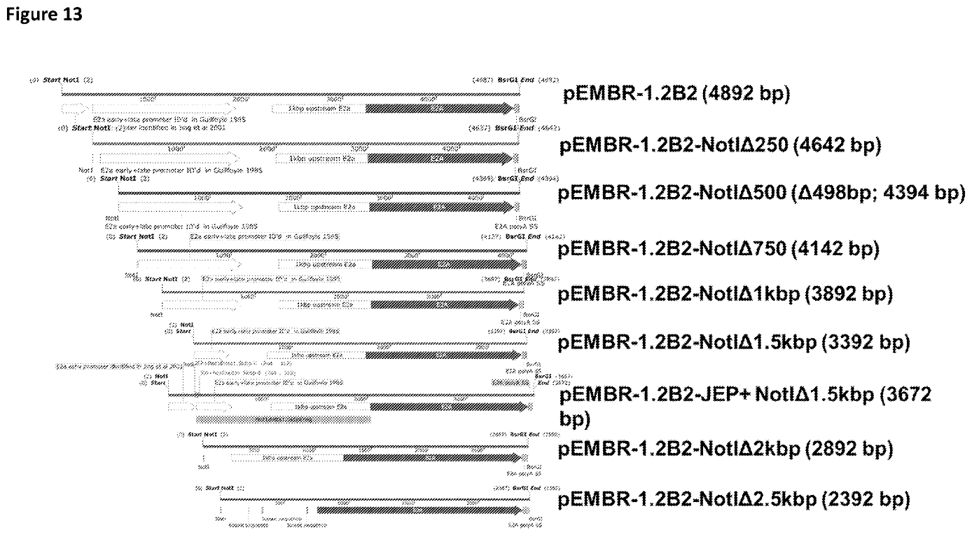

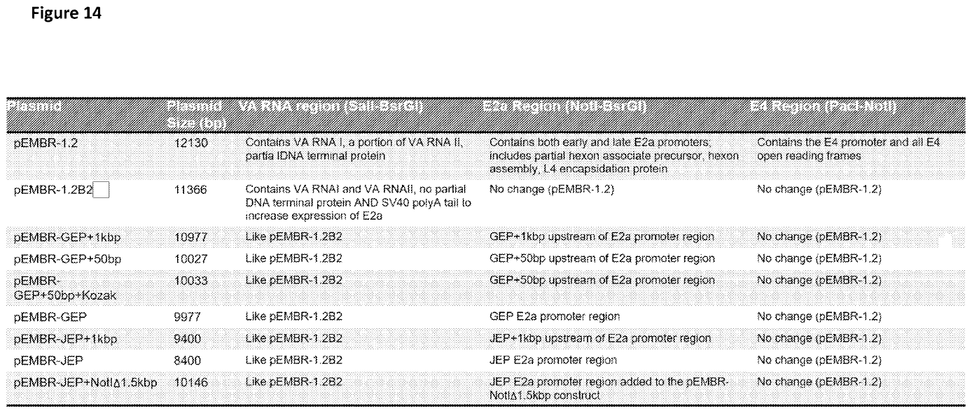

In order to further reduce the size of adenoviral helper plasmids (e.g., those as described herein), adenoviral helper plasmids were designed with modifications to the E2a region. These plasmids were designed to contain the VA RNA region from pEMBR-1.2B2 (SEQ ID NO: 70) and the E4 region from pEMBR-1.2 (SEQ ID NO: 41) with various modifications to the E2a region based on the promoter regions identified by Guilfoyle (GEP) and Jing (JEP) (See FIG. 14). The sequence modifications to the E2a region based on the GEP and JEP promoters are depicted in FIG. 12 and the sequence modifications to the E2a region based on truncations to the NotI end of the pEMBR1.2B2 E2a region are depicted in FIG. 13.

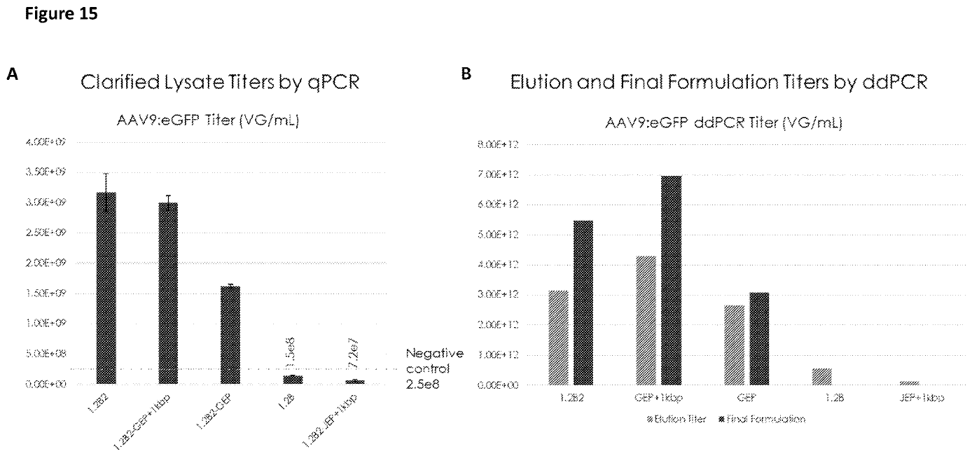

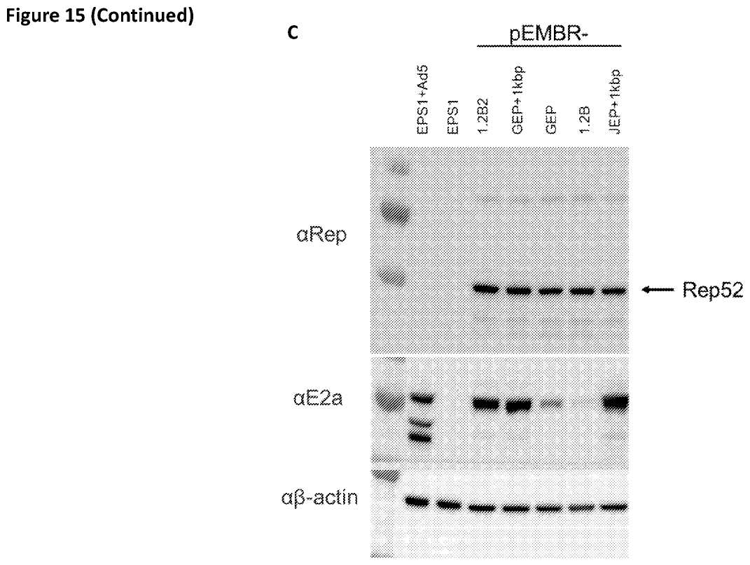

These plasmids were tested for the ability to produce AAV in a large volume of 500 mL culture (See FIG. 15A-B). In large volume, pEMR-1.2B2-GEP+1 kbp (SEQ ID NO: 81) outperformed pEMBR-1.2B2-GEP (SEQ ID NO: 80). Rep52 and E2a protein expression was also assessed in these plasmids (See FIG. 15C). These data demonstrate that Rep52 is present at a similar level in all constructs tested, and E2a protein expression is likely correlated with AAV production. Although the JEP+1 kbp plasmid had E2a protein produced at normal levels, it is missing sequence encoding the L4 (33K and 22K) region.

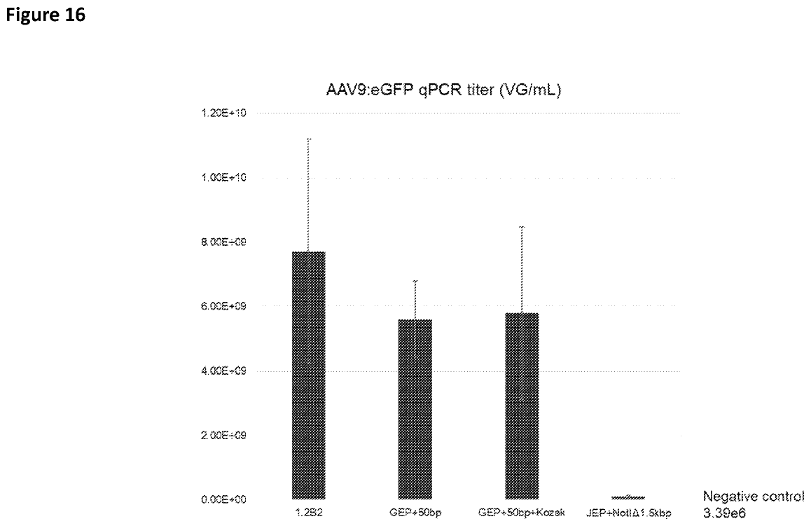

Further testing was performed to analyze AAV production with various constructs in 75 mL culture (See FIG. 16). These data show that there is likely little difference in AAV production between pEMBR-1.2B2 (SEQ ID NO:70), pEMBR-1.2B2-GEP+50 bp (SEQ ID NO: 82), and pEMBR-1.2B2-GEP+50 bp+Kozak (SEQ ID NO: 83). The JEP+NotIΔ1.5 kbp which does not contain the GEP promoter nor L4 (33K and 22K) region produced AAV at a low level.

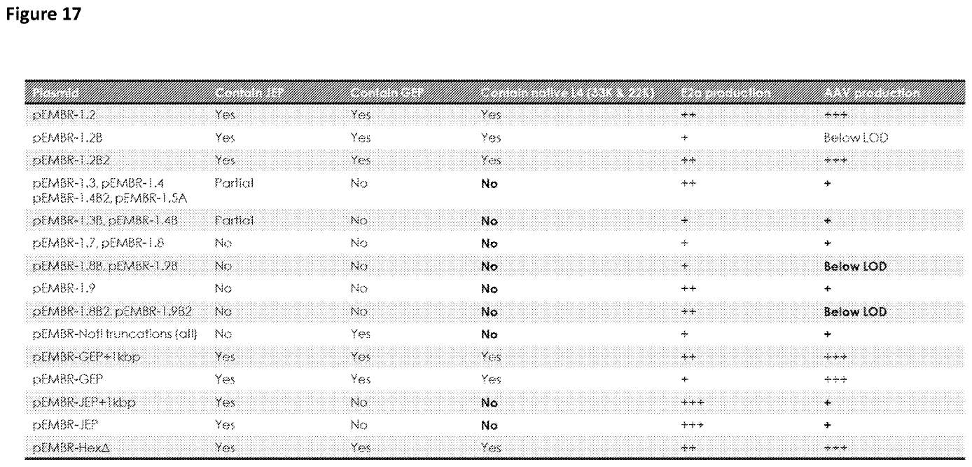

A table depicting which elements are included in various plasmids (e.g., those as described herein) and ability to produce E2a protein and AAV is shown in FIG. 17. Without wishing to be bound by any particular theory, these data indicate that the L4 region (containing 33K and 22K ORFs). JEP/GEP regional requirements. and adequate E2a production is important for the production of AAV.

### Example 4: Exemplary Engineered Adenoviral Helper Plasmids to Reduce Plasmid Size while Maintaining Production of AAV

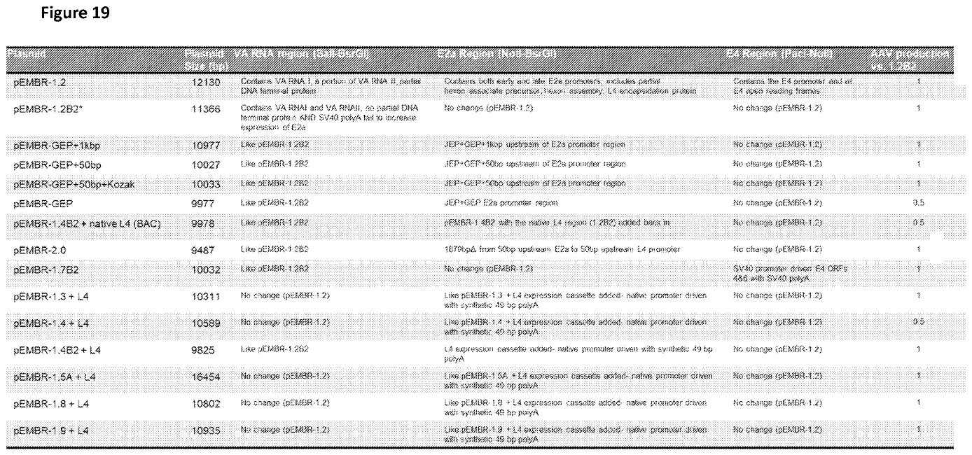

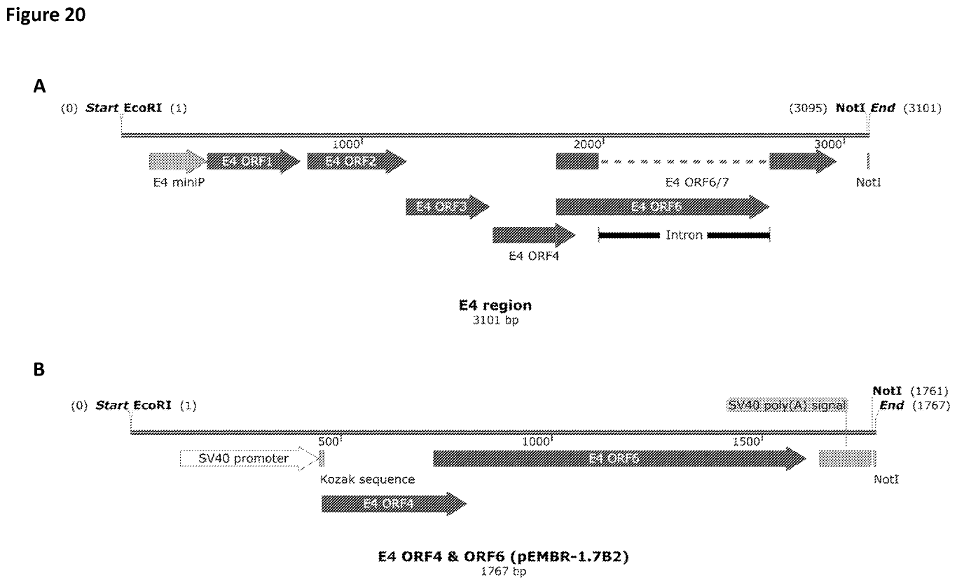

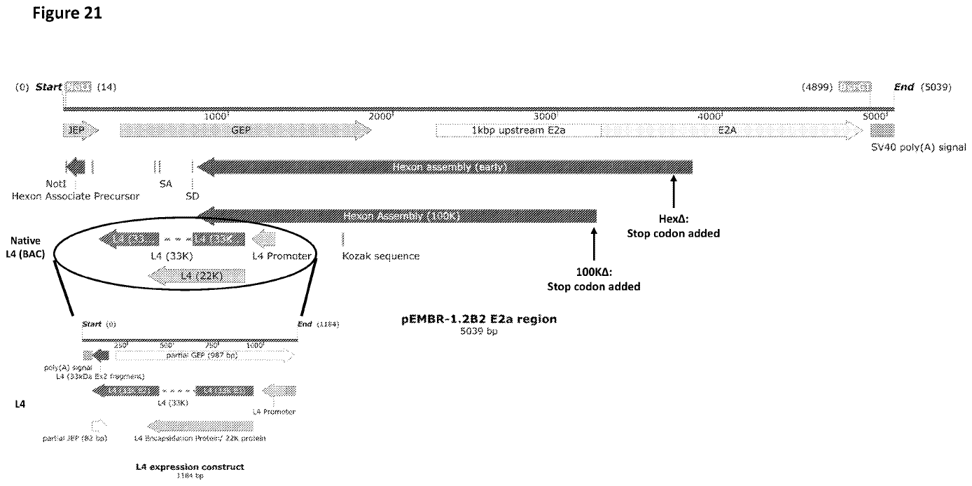

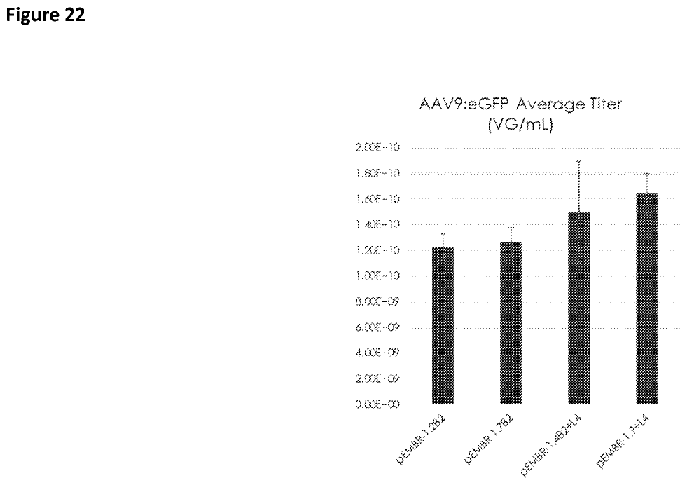

In order to further reduce the size of adenoviral helper plasmids (e.g., those as described herein), adenoviral helper plasmids were designed with modifications to the L4 region, in particular. These plasmids were designed with various modifications to the L4 region (See FIGS. 18-19). The sequence modifications to the L4 region are depicted in FIG. 21. The ability of these plasmids to produce AAV from 75 mL culture were assessed (See FIG. 22). Without wishing to be bound by any particular theory, these data demonstrate that only a construct comprising nucleotide sequence for E4 ORFs 4&6 may be required to maintain AAV production. Without wishing to be bound by any particular theory, these data also demonstrate that adding the sequence for the L4 region (containing 33K and 22K ORFs) with the synthetic polyA tail rescues AAV production in constructs which previously did not produce AAV.

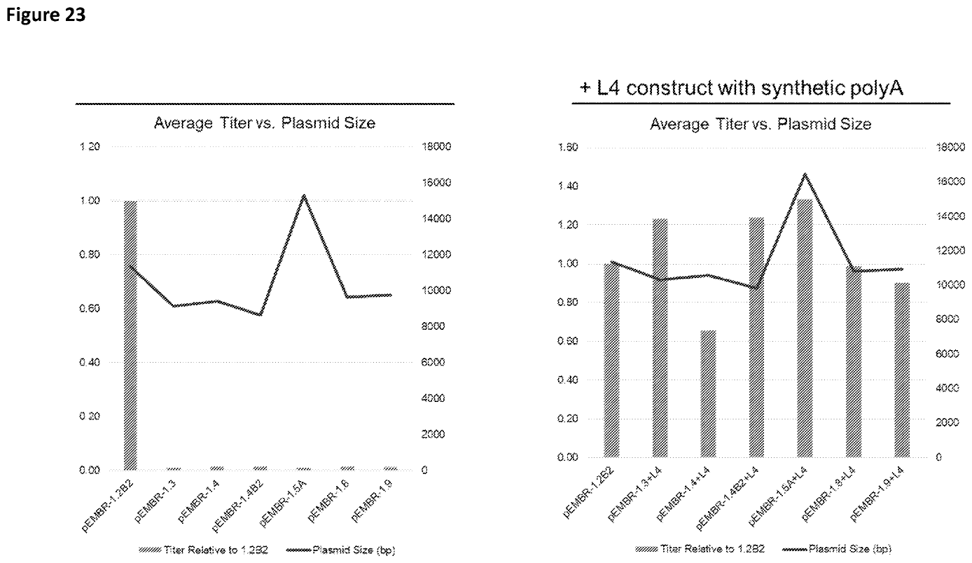

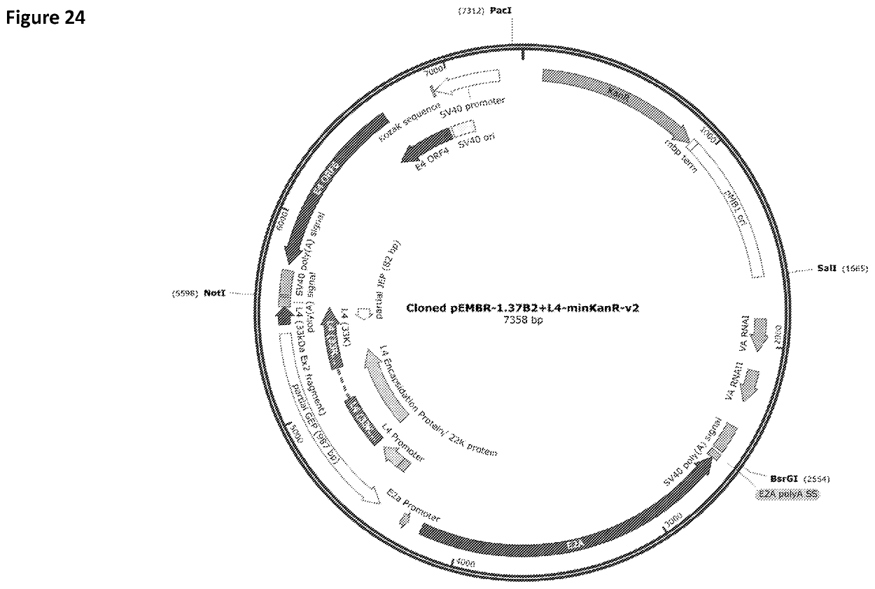

Further testing was performed to analyze AAV production in various constructs with and without the L4 region (containing 33K and 22K ORFs) with the synthetic polyA tail, in comparison to AAV production with pEMBR-1.2B2 where plasmid size is also shown (See FIG. 23). The addition of the L4 region (containing 33K and 22K ORFs) with the synthetic polyA tail to plasmids which previously did not produce AAV resulted in rescued AAV production. An exemplary adenoviral plasmid map is depicted in FIG. 24 which has been engineered (e.g., with modifications as described herein) to reduce plasmid size while maintaining production of AAV.

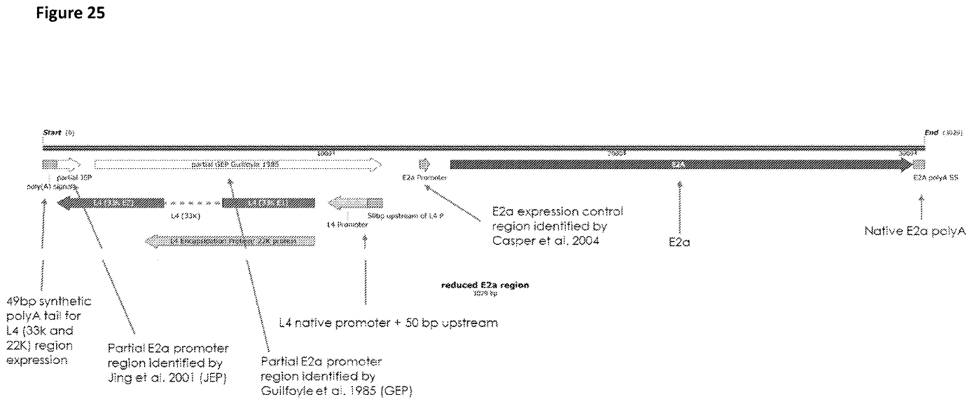

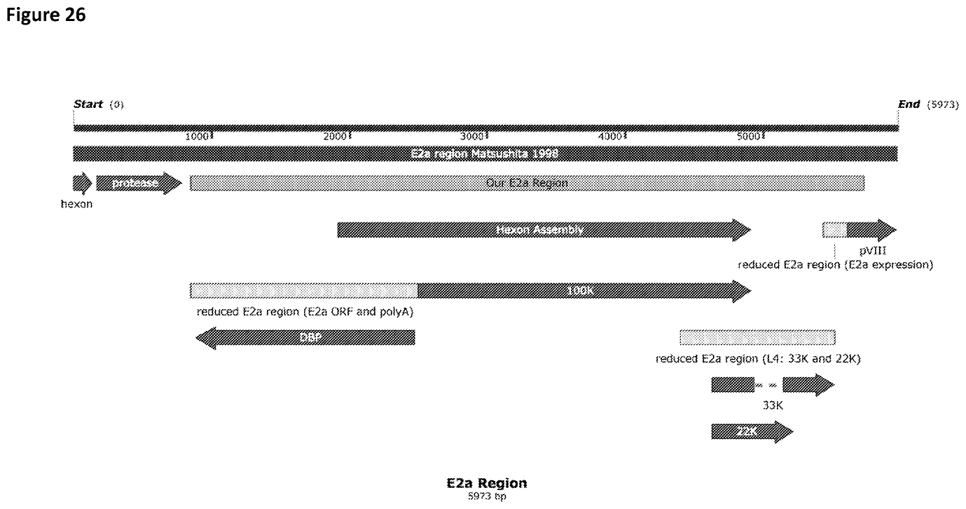

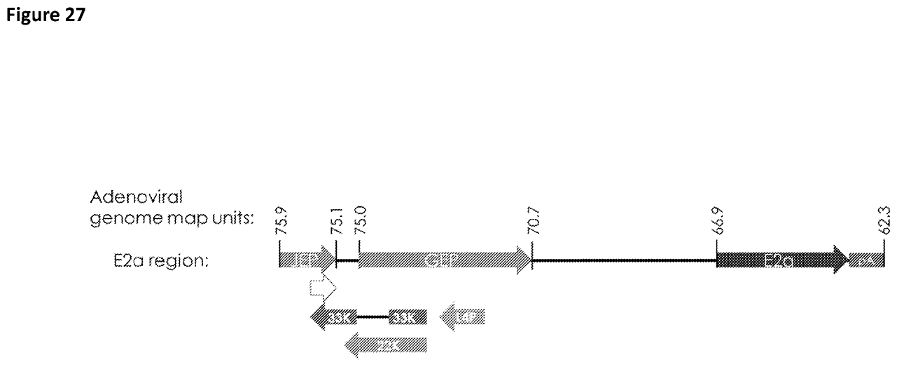

Additionally, this Example describes adenoviral helper plasmids that have been engineered to reduce plasmid size while maintaining production of AAV. The E2a region has been engineered in plasmids described in this example to include native E2a ORF and native E2a poly A tail, the L4 region consisting of 50 bp upstream of native L4 promoter, native L4 promoter, and 33 kDa and 22 kDa ORFs (See FIG. 25). A fragment of the L4 33 kDa Ex 2 comprising an E2a promoter region has been repeated for E2a expression. The E2a region has been engineered in plasmids described in this example to not contain L3 23 kDa viral endoprotease. Hexon Assembly, L4 100K region, nor L4 pVIII Hexon Associated Precursor proteins.

### Example 5: Introduction of Supplementary Accessory Genes to Modified Adenoviral Helper Plasmids

This Example describes the removal of adenoviral helper genes which results in a smaller adenoviral helper plasmid and allows for the addition of supplementary genes to further improve AAV quality and yield. Specifically, various pEMBR plasmids of various sizes and comprising various supplementary genes (e.g., UL30, UL42, etc.) will be designed from pEMBR backbone plasmids (e.g., those as described herein) and tested AAV production. The HSV-1 DNA polymerase genes (UL30 and UL42) will be added back to plasmids to help replicate the AAV transgene, even when cells are not in S phase. The supplementary genes will be designed to be made as a single transcript. Any number of promoters could be used, including CBA, CMV, PGK, etc. and any number of polyA sites could be used. The direction in which the construct is cloned into the plasmid should theoretically not affect expression because this region will contain both promoter and polyA signal which induces expression of supplementary genes independent from the rest of the plasmid.

Vector yields for AAV (e.g., AAV9) in clarified lysate will be measured by qPCR with various pEMBR plasmids designed to comprise various supplementary genes (e.g., UL30, UL42, etc.; See pEMBR-1.5A+L4 in FIG. 23).

### Example 6: Provision of L4 Region Sequence in Trans Rescues AAV Production

This Example demonstrates that provision of L4 region sequences, e.g., L4 33K and/or L4 22K, in trans (i.e., not included in the adenoviral helper plasmid containing VA RNA. an E4 region, and an E2a region) can rescue and/or increase production of AAV.

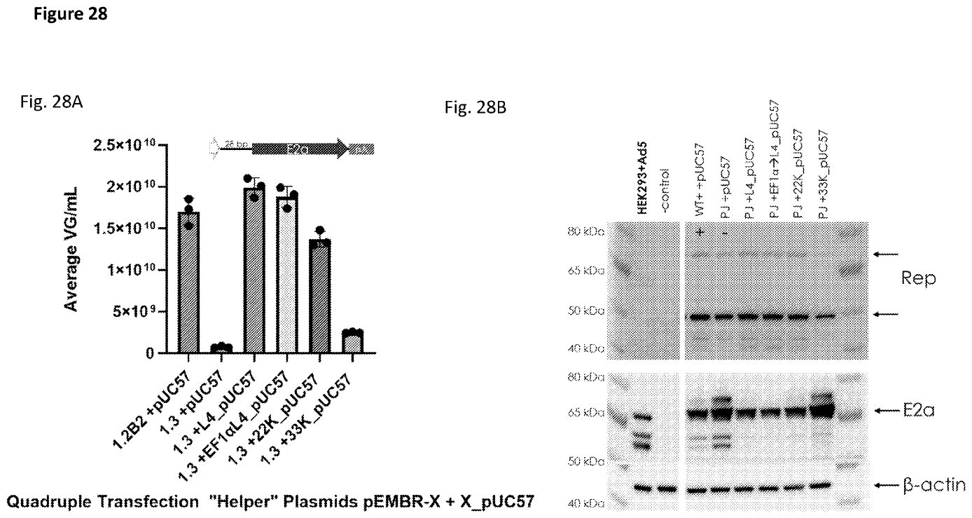

In the present example, a quadruple transfection was performed to add the L4 trans plasmid encoding the L4 region to cells in trans. Adding the L4 region in trans restored rAAV production to wild-type (pEMBR-1.2B2) levels regardless of whether the native promoter driven L4 (L4_pUC57) or an exogenously driven (by human elongation factor 1 alpha, Ef1αL4_pUC57) L4 was added (FIG. 28A).

To determine which L4 protein was more essential, we also utilized plasmids with natively driven codon-optimized 22K or 33K (22K_pUC57 and 33K_pUC57). Our results indicated that 22K was more essential to rAAV production; however, it appeared that the L4 region proteins' effect on production was additive (FIG. 28A). Analysis of E2a protein by western blot revealed a slight decrease in E2a protein when 22K was present (FIG. 28B).

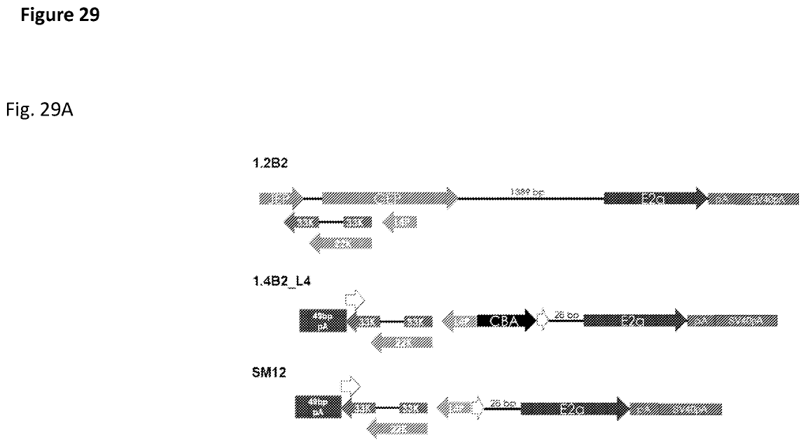

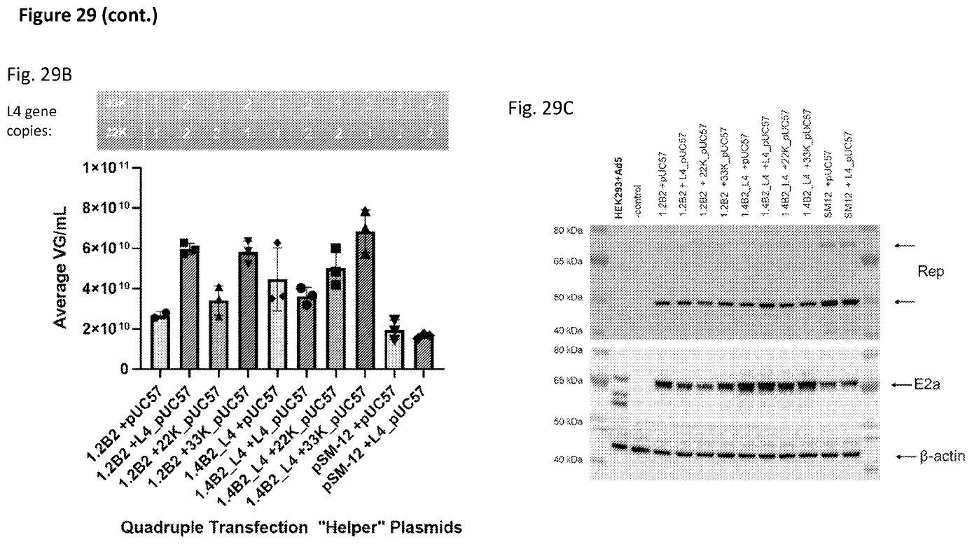

To further test the rescue and/or increase AAV production, L4 gene dosage by adding additional copies of L4 region proteins was performed. The current example used for rAAV production was achieved by quadruple transfection of producer cells. The E2a region of the helper plasmids used in the quadruple transfection are shown in FIG. 29A. FIG. 29B shows average rAAV titer (VG/mL) from triplicate 75 mL quadruple plasmid transfected cultures determined by qPCR for the E2a constructs indicated plus the pUC57 vector containing the specified L4 construct to compare L4 gene dosage of 1X L4 genes, 2X L4 genes, 1X 33K with 2X 22K, and 2X 33K with 1X 22K against the control condition (wild type plus empty vector; 1.2B2+pUC57). Rep and E2a expression levels resulting from the transfections described in FIG. 29B are shown in FIG. 29C. Beta-actin is shown as a loading control. Adenovirus type 5 infected HEK293/HEK293T cells are shown as a positive control and uninfected/nontransfected HEK293 cells are shown as a negative control. Notably, in some conditions the provision of an additional copy of the L4 region (e.g., 1.2B2+L4_pUC57) resulted in increased rAAV titer. Further, provision of additional copies of 33K resulted in increased rAAV titer in several varying conditions. See FIG. 29B.

### Example 7: Table of Sequences

The below Table of Sequences lists and describes the various sequences discussed herein. Unless stated otherwise, all sequences are recited with 5′ to 3′ directionality of the positive strand of a plasmid. This directionality is preserved irrespective of the orientation of a gene or element described to be associated with a sequence. Asterisks as used herein indicate a stop codon.

Lengthy table referenced here
US20260201418A1-20260716-T00001
Please refer to the end of the specification for access instructions.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

LENGTHY TABLES
The patent application contains a lengthy table section. A copy of the table is available in electronic form from the USPTO web site (https://seqdata.uspto.gov/docdetail?docId=US20260201418A1). An electronic copy of the table will also be available from the USPTO upon request and payment of the fee set forth in 37 CFR 1.19(b)(3).

We claim:

1. An adenoviral helper plasmid comprising a nucleotide sequence encoding:
(a) E2a protein;
(b) an E4 region; and
(c) a VA RNA region;
wherein the adenoviral helper plasmid does not comprise a nucleotide sequence encoding one or more of:
a Fiber protein or portion thereof;
a L1-52/55K (Packaging Protein 3); and
a Peripentonal Hexon-Associated Protein.

2. The adenoviral plasmid of claim 1, further comprising an engineered L4 region

3. The adenoviral plasmid of claim 2, wherein the engineered L4 region does not express an L4 100K protein.

4. The adenoviral plasmid of claim 2, wherein the engineered L4 region does not comprise a complete L4 100K encoding nucleotide sequence.

5. The adenoviral plasmid of claim 4, wherein the engineered L4 region does not comprise base pairs 24061-25961 relative to GenBank Accession Number M73260.

6. The adenoviral plasmid of claim 2, wherein the engineered L4 region comprises at least one nucleotide sequence encoding L4-33K at least 80% identical to SEQ ID: NO. 5.

7. The adenoviral plasmid of claim 6, wherein the engineered L4 region comprises two nucleotide sequences encoding L4-33K at least 80% identical to SEQ ID: NO. 5.

8. The adenoviral plasmid of claim 2, wherein the engineered L4 region comprises a nucleotide sequence encoding L4-22K at least 80% identical to SEQ ID: NO. 7.

9. The adenoviral plasmid of claim 2, wherein the engineered L4 region comprises a nucleotide sequence encoding L4-33K and a nucleotide sequence encoding L4-22K.

10. The adenoviral plasmid of claim 9, wherein the engineered L4 region comprises 50 nucleotides upstream of the L4-33K/22K region.

11. The adenoviral plasmid of claim 10, wherein the 50 nucleotides upstream of the L4-33K/22K region is identified relative to the L4-33K/22K region of GenBank Accession Number M73260.

12. The adenoviral plasmid of claim 11, further comprising a synthetic polyA signal downstream of the L4-33K/22K region.

13. The adenoviral plasmid of claim 1, wherein the adenoviral helper plasmid does not comprise a nucleotide sequence encoding L3 23 kDa viral endoprotease.

14. The adenoviral plasmid of claim 1, wherein the adenoviral helper plasmid does not comprise a nucleotide sequence encoding L4 pVIII.

15. The adenoviral helper plasmid of claim 9, wherein the engineered L4 region comprises a nucleotide sequence comprising an E2a promoter region upstream of the nucleotide sequence encoding L4-33K and the nucleotide sequence encoding L4-22K.

16. The adenoviral helper plasmid of claim 1, wherein the adenoviral helper plasmid does not comprise a nucleotide sequence encoding DNA Terminal Protein.

17. The adenoviral helper plasmid of claim 1, wherein expression of E2a protein is under the control of an E2a promoter.

18. The adenoviral helper plasmid of claim 1, wherein expression of E2a protein is under the control of an E2a promoter and a chicken D-actin promoter, wherein the chicken D-actin promoter is upstream of the E2a promoter.

19. The adenoviral helper plasmid of claim 1, wherein expression of E2a protein is under the control of the chicken D-actin promoter.

20. The adenoviral helper plasmid of claim 18 or 19, wherein in the chicken β-actin promoter has a nucleotide sequence that is at least 80% identical to SEQ ID. No: 26.

21. The adenoviral helper plasmid of claim 1, wherein the adenoviral helper plasmid comprises an E2a polyadenylation signal downstream of E2a.

22. The adenoviral helper plasmid of claim 1, wherein the adenoviral helper plasmid contains an SV40 polyadenylation signal downstream of E2a.

23. The adenoviral helper plasmid of claim 20, wherein the SV40 polyadenylation signal is downstream of an E2a polyadenylation signal.

24. The adenoviral helper plasmid of claim 21 or 20, wherein the SV40 polyadenylation signal has a sequence that is at least 80% identical to SEQ ID. No: 28.

25. The adenoviral helper plasmid of claim 1, wherein the adenoviral helper plasmid further comprises nucleotide sequences encoding HSV-1 UL30 and HSV-1 UL42,
wherein at least one of the nucleotide sequences is at least 80% identical to SEQ ID NO: 29;
wherein at least one of the nucleotide sequences is at least 80% identical to SEQ ID NO: 31;
wherein UL30 and UL42 are separated by a P2A cleavage site encoded by a nucleic acid sequence that is at least 80% identical to SEQ ID NO: 33.

26. The adenoviral helper plasmid of claim 1, wherein the adenoviral helper plasmid further comprises nucleotide sequences encoding HSV-1 UL29/ICP8.

27. The adenoviral helper plasmid of any one of claim 23 and XX, wherein the HSV-1 UL30; UL42; and UL29 are separated by a P2A cleavage site encoded by a nucleic acid sequence that is at least 80% identical to SEQ ID NO: 139

28. The adenoviral helper plasmid of claim 1, wherein the E4 region does not comprise E4orf1, and wherein the E4 region does not comprise E4orf2.

29. The adenoviral helper plasmid of claim 1, wherein the E4 region is operably linked to the E4 mini promoter, wherein the E4 mini promoter has a nucleotide sequence that is at least 80% identical to SEQ ID NO: 1.

30. The adenoviral helper plasmid of claim 1, wherein the E4 region is operably linked to the SV40 promoter, wherein the SV40 promoter has a nucleotide sequence that is at least 80% identical to SEQ ID NO: 2.

31. The adenoviral helper plasmid of any of the above claims, wherein the adenoviral helper plasmid comprises a resistance gene.

32. The adenoviral helper plasmid of claim 31, wherein the resistance cassette is a kanamycin resistance gene.

33. An adenoviral helper plasmid having 80% sequence identity to any one of SEQ ID NO: 80-105.

34. An adenoviral helper plasmid comprising an engineered E2A region comprising any one of nucleotide sequences SEQ ID NO: 106 or SEQ ID NO: 112.

35. The adenoviral helper plasmid of claim 1 or claim 2, wherein the adenoviral helper plasmid does not comprise a nucleotide sequence encoding L4 33K.

36. The adenoviral helper plasmid of claim 1 or claim 2, wherein the adenoviral helper plasmid does not comprise a nucleotide sequence encoding L4 22K.

37. The adenoviral helper plasmid of claim 1 or claim 2, wherein the adenoviral helper plasmid does not comprise a nucleotide sequence encoding L4 22K and does not comprise a nucleotide sequence encoding L4 33K.

38. An L4 trans plasmid comprising a nucleotide sequence encoding an engineered L4 region.

39. The L4 trans plasmid of claim 38, wherein the engineered L4 region comprises a nucleotide sequence encoding L4 at least 80% identical to SEQ ID NO. 115.

40. The L4 trans plasmid of claim 38, wherein the engineered L4 region comprises a nucleotide sequence encoding L4-33K at least 80% identical to SEQ ID NO. 120.

41. The L4 trans plasmid of claim 38, wherein the engineered L4 region comprises a nucleotide sequence encoding L4-22K at least 80% identical to SEQ ID NO. 7.

42. A composition comprising:
an adenoviral helper plasmid of any one of claims 35, 36, or 37; and
an L4 trans plasmid of any one of claims 38-41.

43. A method of producing a recombinant adenoviral associated viral vector comprising:
transfecting a producer cell with an AAV vector plasmid, an AAV Rep-Cap expressing plasmid, and the adenoviral helper plasmid of any one of claims 1-37.

44. The method of claim 43, wherein the AAV vector plasmid comprises AAV inverted terminal repeats (ITRs) and a transgene of interest.

45. The method of claim 43, wherein the method further comprises transfecting a producer cell with an L4 trans plasmid of any one of claims 38-41.

46. The method of any one of claim 43-45 wherein both the L4 trans plasmid and the adenoviral helper plasmid comprise an engineered L4 region.

47. The method of claim 46, wherein the engineered L4 region of the L4 trans plasmid comprises a nucleotide sequence encoding L33K.

48. A method of producing a recombinant adenoviral associated viral vector comprising:
transfecting a producer cell with an AAV vector plasmid and the adenoviral helper plasmid of any one of claims 1-37, wherein the producer cell stably expresses Rep-Cap and comprises a nucleotide sequence encoding an L4 region or portion thereof.

49. The method of claim 48, wherein the AAV vector plasmid comprises AAV inverted terminal repeats (ITRs) and a transgene of interest.

50. The method of claim 49, wherein the method further comprises transfecting a producer cell with an L4 trans plasmid of any one of claims 38-41.

51. A method of producing a recombinant adenoviral associated viral vector comprising:
transfecting a producer cell with an AAV vector plasmid, an AAV Rep-Cap expressing plasmid, the adenoviral helper plasmid of any one of claims 1-37, and an L4 trans plasmid.

52. The method of 51 wherein, the L4 trans plasmid comprises a nucleotide sequence encoding L33K.

53. The method of 51 wherein, the producer cell a nucleotide sequence encoding an L4 region or portion thereof.
